(19) United States
(12) Patent Application Publication
(10) Pub. No.: US 2009/0074696 A1
(43) Pub. Date: Mar. 19, 2009

(54) COSMETIC COMPOSITION COMPRISING A CATIONIC COPOLYMER AND A STARCH AND COSMETIC TREATMENT PROCESS THEREOF

(76) Inventors: Olga BIGANSKA, Asnieres sur Seine (FR); Laurent Chesneau, Levallois Perret (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/210,791

(22) Filed: Sep. 15, 2008

Related U.S. Application Data
(60) Provisional application No. 60/960,230, filed on Sep. 21, 2007.

(30) Foreign Application Priority Data
Sep. 14, 2007 (FR) 0757584

(51) Int. Cl. A61K 8/81; A61Q 5/02; A61Q 5/12

(52) U.S. Cl. 424/70.13; 514/772.4; 424/78.02; 510/119

(57) ABSTRACT

The disclosure relates to novel cosmetic compositions comprising, in a cosmetically acceptable medium:
(i) at least one cationic polymer which is obtained by polymerization of a mixture of monomers comprising:
   a) at least one vinyl monomer substituted with at least one amino group,
   b) at least one hydrophobic nonionic vinyl monomer, chosen from those of formulae (I) and (II):

CH₂═C(X)Z, (I)

CH₂═CH—OC(O)R; (II)
wherein X, Z, and R are defined in the present specification;
c) at least one associative vinyl monomer, and
d) at least one hydroxylated nonionic vinyl monomer; and
(ii) at least one starch.

These compositions may have a texture which does not change over time and which may allow a better distribution, better lathering properties and also better cosmetic properties.

This application claims benefit of U.S. Provisional Application No. 60/960,230, filed Sep. 21, 2007, the contents of which are incorporated herein by reference. This application also claims benefit of priority under 35 U.S.C. §119 to French Patent Application No. FR 0757584, filed Sep. 14, 2007, the contents of which are also incorporated herein by reference.

The present disclosure relates to a cosmetic composition, for example, for conditioning the hair, comprising at least one modified or unmodified starch and at least one cationic copolymer, and to a cosmetic treatment process for keratin materials, such as the hair.

A shampoo or conditioner is most commonly in the form of a more or less viscous liquid. Products of which the texture is sufficiently thick to remain on the hair, without running, for a certain period of time, are sought. This thicker, or even gel, texture, should not impair the qualities of said product when used.

The thickening and/or gelling of aqueous media with polymers has for a long time been an important subject of cosmetic research. Obtaining an advantageous thickening effect with a water-soluble polymer generally assumes a high molar mass and a large hydrodynamic volume. The gelling of an aqueous medium is then considered to be the result of a three-dimensional polymer network obtained by crosslinking of linear polymers or by copolymerization of bifunctional and polyfunctional monomers. However, the use of such polymers of very high molar mass poses a certain number of problems, such as the relatively unpleasant texture and the difficult spreading of the gels obtained.

Cosmetic compositions thickened with thickening polysaccharides, such as starch and celluloses, have already been proposed for the treatment of keratin materials, such as the hair.

However, such compositions have drawbacks such as rinseability problems, stability problems, such as syneresis problems, difficulties in distributing them over keratin materials, and also insufficient cosmetic properties.

In summary, current cosmetic conditioning compositions comprising starch are not entirely satisfactory. Thus, it is sought to obtain cosmetic compositions having very good cosmetic properties, for example on very sensitized hair, good properties for use (good distribution, lather, good rinsing) and also good stability.

Applicants have now discovered that the combination of a starch and of a particular cationic copolymer makes it possible to remedy these drawbacks.

The compositions according to the disclosure may give the hair more mass, more body, and more sheen compared with compositions comprising only a starch.

The compositions according to the disclosure may give the hair more manageability (control), more smoothness, and suppleness compared with compositions comprising only a cellulose.

The hair may be more supple during rinsing.

These compositions may have a texture which changes little or not at all over time at ambient temperature (25° C.) and at 45° C. The stability of the compositions may therefore be satisfactory at ambient temperature and at 45° C.; for example, the compositions according to the disclosure may no longer exhibit the syneresis effect. These compositions may allow a better distribution of the product during application. When the compositions further comprise foaming surfactants, their lathering properties, for example the start of lathering, may be improved.

Thus, the present disclosure now provides new cosmetic compositions comprising, in a cosmetically acceptable aqueous medium:
(i) at least one cationic polymer which is produced by the polymerization of a mixture of monomers comprising:
   a) at least one vinyl monomer substituted with at least one amino group,
   b) at least one hydrophobic nonionic vinyl monomer chosen from those of formulae (I) and (II):

CH₂═C(X)Z (I)

CH₂═CH—OC(O)R (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₆, —C₆H₄R¹, —C₆H₄OR¹, —C₆H₄Cl, —CN, —NHC(O)CH₃, —NHC(O)H, —N-(2-pyrrolidonyl), —N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group;
each R¹ independently is chosen from a C₁-C₃₀ alkyl group, a hydroxylated C₂-C₃₀ alkyl group, and a halogenated C₁-C₃₀ alkyl group;
c) at least one associative vinyl monomer, and
d) at least one hydroxylated nonionic vinyl monomer; and
(ii) at least one starch.

Another embodiment of the disclosure comprises a cosmetic treatment process for keratin materials, such as the hair, using the abovementioned composition.

A further embodiment of the disclosure is the use of said composition as a shampoo, and for example as a conditioner.

Other embodiments, features, aspects and advantages of the disclosure will emerge more clearly on reading the description and the various examples which follow.

The term “sensitized hair” is understood to mean, according to the present disclosure, hair that has undergone outside physical attacks (e.g., light, heat, waves, etc.), mechanical attacks (e.g., repeated blow-drying, combing or brushing, etc.) and/or chemical attacks (e.g., oxidation dyeing, bleaching, permanent-waving, hair straightening, etc.), Among these attacks, the particularly harmful nature of chemical attacks will be noted. The compositions according to the disclosure may be effective on hair sensitized by chemical attacks.

The term “cosmetically acceptable medium” is intended to mean a medium compatible with all keratin materials such as the skin, the hair, the nails, the eyelashes, the eyebrows, the lips, and any other area of the body or the face.

One of the features of the disclosure is the presence of at least one cationic polymer which is obtained by polymerization of a mixture of monomers comprising a) at least one vinyl monomer substituted with at least one amine group, b) at least one hydrophobic nonionic vinyl monomer, c) at least one associative vinyl monomer, and d) at least one hydroxylated nonionic vinyl monomer. In at least one embodiment, the monomers constituting the at least one cationic copolymer also comprise at least one semi-hydrophobic surfactant vinyl monomer (e). The monomers a) to e) are different from one another.

In at least one embodiment, the at least one cationic polymer (i) is a thickening polymer.

For the purpose of the present disclosure, the term “thickening polymer” is intended to mean a polymer which, when introduced at 1% by weight into an aqueous or aqueous-alcoholic solution comprising 30% by weight of ethanol, and at pH 7, makes it possible to attain a viscosity of at least 100 cps at 25° C., and a shear rate of 1 s⁻¹. This viscosity can be measured using a viscometer with cone-plate geometry, for example, a Haake RS 600 rheometer. For example, these polymers may make it possible to increase the viscosity of the compositions which comprise them by at least 50 cps at 25° C. and at 1 s⁻¹.

The at least one cationic polymer (i) used in the composition according to the disclosure, and the process for producing it, are described, for example, in international application WO 2004/024779.

For the purpose of the present disclosure, the term “vinyl monomer” is intended to mean a monomer comprising at least one R₀CH═C(R₀)— group, wherein each R₀ is independently chosen from a hydrogen atom, a C₁-C₃₀ alkyl, —C(O)OH, —C(O)OR′₀, —O—C(O)OR′₀, —C(O)NHR′₀, and —C(O)NR′₀R″₀, wherein R′₀ and R″₀, which may be identical or different, are C₁-C₃₀ alkyl groups.

Thus, for example, for the purpose of the present disclosure, (meth)acrylates and (meth)acrylamides are vinyl monomers.

As previously explained, the mixture of monomers for the preparation of the at least one cationic polymer (i) used in the composition according to the disclosure comprises at least one vinyl monomer substituted with at least one amino group.

The at least vinyl monomer substituted with at least one amino group that can be used for the preparation of the at least one cationic polymer (i) used according to the disclosure are basic and polymerizable ethylenically unsaturated monomers. The at least amine group may derive from mono-, di- or polyaminated alkyl groups, or from heteroaromatic groups comprising a nitrogen atom. The at least amine group may be a primary, secondary, or tertiary amine. These monomers may be used in the amine form or in the salt form.

In at least one embodiment, the at least vinyl monomer substituted with at least one amine group is chosen from:
mono(C₁-C₄)alkylamino(C₁-C₈)alkyl (meth)acrylates,
di(C₁-C₄)alkylamino(C₁-C₈)alkyl (meth)acrylates, such as di(C₁-C₄)alkylamino(C₁-C₆)alkyl (meth)acrylates,
mono(C₁-C₄)alkylamino(C₁-C₈)alkyl (meth)acrylamides,
di(C₁-C₄)alkylamino(C₁-C₈)alkyl(meth)acrylamides,
(meth)acrylamides comprising a heterocyclic group or groups comprising a nitrogen atom,
(meth)acrylates comprising a heterocyclic group or groups comprising a nitrogen atom,
nitrogenous heterocycles comprising a vinyl group or groups,
   and mixtures thereof.

With respect to the at least one vinyl monomer substituted with at least one amino group, non-limiting mention may be made of:
mono- or di(C₁-C₄ alkyl)amino(C₁-C₄ alkyl) (meth)acrylates, such as 2-(N,N-dimethylamino)ethyl (meth)acrylate, 3-(N,N-dimethylamino) propyl (meth)acrylate, 4-(N,N-dimethylamino)butyl (meth)acrylate, (N,N-dimethylamino)-t-butyl (meth)acrylate, 2-(N,N-diethylamino)ethyl (meth)acrylate, 3-(N,N-diethylamino)propyl (meth)acrylate, 4-(N,N-diethylamino)butyl (meth)acrylate, 2-(N,N-dipropylamino)ethyl (meth)acrylate, 3-(N,N-dipropylamino)propyl (meth)acrylate, and 4-(N,N-dipropylamino)butyl (meth)acrylate;
mono- or di(C₁-C₄ alkyl)amino(C₁-C₄ alkyl)(meth)acrylamides such as N′-(2-N,N-dimethylamino)ethyl(meth)acrylamide and N′-(3-N,N-dimethylamino)propylacrylamide;
(meth)acrylamides or (meth)acrylates comprising a heterocyclic group comprising a nitrogen atom, such as N-(2-pyridyl)acrylamide, N-(2-imidazolyl)methacrylamide, 2-(4-morpholinyl)ethyl methacrylate, 2-(4-morpholinyl)ethyl acrylate, N-(4-morpholinyl)methacrylamide, and N-(4-morpholinyl)acrylamide; and
nitrogenous heterocycles comprising at least one vinyl group, such as 2-vinylpyridine and 4-vinylpyridine.

When the monomers are in the form of salts, they may be mineral salts, such as hydrochloride, sulphate and phosphate salts, or salts of organic acids, such as acetate, maleate and fumarate salts.

Non-limiting examples of the at least one vinyl monomer substituted with at least one amino group include:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate.

The at least vinyl monomer substituted with at least one amino group may be present in an amount ranging from 10% to 70% by weight, such as from 20% to 60% by weight, and for example from 30% to 40% by weight, relative to the total weight of the mixture of monomers.

As previously explained, the mixture of monomers for the preparation of the at least one cationic polymer (i) according to the disclosure also comprises at least one hydrophobic nonionic vinyl monomers b).

The at least one hydrophobic nonionic vinyl monomer that can be used for the preparation of the at least one cationic polymer (i) used according to the disclosure may be chosen from compounds of formulae (I) and (II):

CH₂═C(X)Z, (I)

CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, —N-(2-pyrrolidonyl), —N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group;
each R¹ independently is chosen from a C₁-C₃₀ alkyl group, a hydroxylated C₂-C₃₀ alkyl group, and a halogenated C₁-C₃₀ alkyl group.

Non-limiting mention may be made of C₁-C₃₀ alkyl (meth)acrylates; (C₁-C₃₀ alkyl)(meth)acrylamides; styrene, substituted styrenes and for example vinyltoluene (or 2-methylstyrene), butylstyrene, isopropylstyrene, para-chlorostyrene; vinyl esters, and for example vinyl acetate, vinyl butyrate, vinyl caprolate, vinyl pivalate and vinyl neodecanoate; unsaturated nitriles, and for example (meth)acrylonitrile and acrylonitrile; and unsaturated silanes, and for example trimethylvinylsilane, dimethylethylvinylsilane, allyldimethylphenylsilane, allyltrimethylsilane, 3-acrylamidopropyltrimethylsilane, and 3-trimethylsilylpropyl methacrylate.

In at least one embodiment, the at least one hydrophobic nonionic vinyl monomer is chosen from C₁-C₃₀ alkyl acrylates, C₁-C₃₀ alkyl methacrylates, and mixtures thereof, such as ethyl acrylate, methyl methacrylate or 3,3,5-trimethylcyclohexyl methacrylate.

The at least one hydrophobic nonionic vinyl monomer may be present in an amount ranging from 20% to 80% by weight, such as from 20% to 70% by weight, and for example from 50% to 65% by weight, relative to the total weight of the mixture of monomers.

The at least one associative vinyl monomer that can be used for the preparation of the at least one cationic polymer (i) according to the disclosure may be chosen from compounds having an end (i)′ comprising at least one ethylenic unsaturation for addition polymerization with other monomers of the system, a central polyoxyalkylene portion (ii)′ for conferring selective hydrophilic properties on the polymers, and a hydrophobic end (iii)′ for conferring selective hydrophobic properties on the polymers.

The end (i)′ comprising at least one ethylenic unsaturation of the at least one associative vinyl monomer is, in at least one embodiment, derived from a monocarboxylic or dicarboxylic acid or anhydride comprising at least one α,β-ethylenic unsaturation, such as a C₃- or C₄-monocarboxylic or dicarboxylic acid or anhydride. Alternatively, the end (i)′ of the at least one associative monomer may be derived from an allyl ether or from a vinyl ether; from a nonionic urethane monomer substituted with a vinyl group, as disclosed in reissue U.S. Pat. No. 33,156 or in U.S. Pat. No. 5,294,692; or from a urea reaction product substituted with a vinyl group, as disclosed in U.S. Pat. No. 5,011,978.

The central portion (ii)′ of the at least one associative vinyl monomer is, in at least one embodiment, a polyoxyalkylene segment comprising from 5 to 250, such as from 10 to 120, and for example from 15 to 60 C₂-C₇ alkylene oxide units. In another embodiment, central portions (ii)′ are polyoxyethylene, polyoxypropylene and polyoxybutylene segments comprising from 5 to 150, such as from 10 to 100, and for example from 15 to 60 ethylene oxide, propylene oxide or butylene oxide units, and random or nonrandom blocks of ethylene oxide, propylene oxide or butylene oxide units. In a further embodiment, the central portions (ii)′ are polyoxyethylene segments.

The hydrophobic end (iii)′ of the at least one associative monomer is, in at least one embodiment, a hydrocarbon-based fragment belonging to one of the following hydrocarbon-based categories: a linear C₈-C₄₀ alkyl, a C₂-C₄₀ alkyl substituted with an aryl group, a phenyl substituted with a C₂-C₄₀ alkyl group, a branched C₈-C₄₀ alkyl, a C₈-C₄₀ alicyclic group, and a C₈-C₈₀ complex ester.

For the purpose of the present disclosure, the term “complex ester” is intended to mean any ester other than a simple ester.

For the purpose of the present disclosure, the term “simple ester” is intended to mean any unsubstituted, linear or branched, C₁-C₃₀ saturated aliphatic alcohol ester.

Non-limiting examples of the hydrophobic ends (iii)′ of the at least one associative monomer include linear or branched alkyl groups comprising from 8 to 40 carbon atoms, such as (C₈) capryl, (branched C₈) isooctyl, (C₁₀) decyl, (C₁₂) lauryl, (C₁₄) myristyl, (C₁₆) cetyl, (C₁₆-C₁₈) cetearyl, (C₁₈) stearyl, (branched C₁₈) isostearyl, (C₂₀) arachidyl, (C₂₂) behenyl, (C₂₄) lignoceryl, (C₂₆) cerotyl, (C₂₈) montanyl, (C₃₀) melissyl, and (C₃₂) lacceryl.

Non-limiting examples of linear or branched alkyl groups comprising from 8 to 40 carbon atoms and derived from a natural source include alkyl groups derived from hydrogenated groundnut oil, from soybean oil and from canola oil (predominantly C₁₈), hydrogenated C₁₆-C₁₈ tallow oil; and hydrogenated C₁₀-C₃₀ terpenols, such as hydrogenated geraniol (branched C₁₀), hydrogenated farnesol (branched C₁₅), or hydrogenated phytol (branched C₂₀).

Non-limiting examples of phenyls substituted with a C₂-C₄₀ alkyl include octylphenyl, nonylphenyl, decylphenyl, dodecylphenyl, hexadecylphenyl, octadecylphenyl, isooctylphenyl and sec-butylphenyl.

C₈-C₄₀ alicyclic groups may, for example, be groups derived from sterols of animal origin, such as cholesterol, lanosterol or 7-dehydrocholesterol; or derivatives of plant origin, such as phytosterol, stigmasterol or campesterol; or derivatives derived from microorganisms, such as ergosterol or mycrosterol. Other C₈-C₄₀ alicyclic groups that can be used in the present disclosure are, for example, cyclooctyl, cyclododecyl, adamantyl and decahydronaphthyl, and groups derived from natural C₈-C₄₀ alicyclic compounds such as pinene, hydrogenated retinol, camphor and isobornyl alcohol.

The C₂-C₄₀ alkyl groups substituted with an aryl group may, for example, be 2-phenylethyl, 2,4-diphenylbutyl, 2,4,6-triphenylhexyl, 4-phenylbutyl, 2-methyl-2-phenylethyl, or 2,4,6-tri(1′-phenylethyl)phenyl.

Among the C₈-C₄₀ complex esters that can be used as end (iii)′, non-limiting mention may be made of hydrogenated castor oil (for example 12-hydroxystearic acid triglyceride); 1,2-diacylglycerols such as 1,2-distearyl glycerol, 1,2-dipalmityl glycerol or 1,2-dimyristyl glycerol; di-, tri- or polyesters of sugars, such as 3,4,6-tristearyl glucose or 2,3-dilauryl fructose; and sorbitan esters such as those disclosed in U.S. Pat. No. 4,600,761.

The associative vinyl monomers that can be used according to the disclosure may be prepared by any method known from the prior art. Reference may, for example, be made to U.S. Pat. No. 4,421,902, U.S. Pat. No. 4,384,096, U.S. Pat. No. 4,514,552, U.S. Pat. No. 4,600,761, U.S. Pat. No. 4,616,074, U.S. Pat. No. 5,294,692, U.S. Pat. No. 5,292,843, U.S. Pat. No. 5,770,760 and U.S. Pat. No. 5,412,142.

In at least one embodiment, the at least one associative vinyl monomer that can be used according to the disclosure is chosen from the compounds of formula (III):

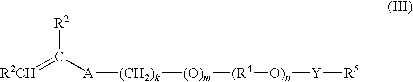

wherein:
each R² is independently chosen from a hydrogen atom, a methyl group, a —C(O)OH group, and a —C(O)OR³ group;
R³ is a C₁-C₃₀ alkyl;
A is chosen from —CH₂C(O)O—, —C(O)O—, —O—, —CH₂O—, —NHC(O)NH—, —C(O)NH—, —Ar—(CE₂)_{z}—NHC(O)O—, —Ar—(CE₂)_{z}-NHC(O)NH—, and —CH₂CH₂—NHC(O)— groups;
Ar is a divalent aryl group;
E is chosen from a hydrogen atom and a methyl group;
z is an integer ranging from 0 to 1;
k is an integer ranging from 0 to 30;
m is an integer ranging from 0 or 1, with the provisos that when k is 0, then m is 0, and when k is an integer ranging from 1 to 30, then m is 1;
(R⁴—O)ₙ is a polyoxyalkylene, which is a homopolymer, a random copolymer, or a block copolymer, comprising C₂-C₄ oxyalkylene units;
R⁴ is chosen from C₂H₄, C₃H₆, C₄H₈, and mixtures thereof;
n is an integer ranging from 5 to 250;
Y is chosen from —R⁴O—, —R⁴NH—, —C(O)—, —C(O)NH—, —R⁴NHC(O)NH—, and —C(O)NHC(O)—;
R⁵ is a substituted or unsubstituted alkyl chosen from linear C₈-C₄₀ alkyls, branched C₈-C₄₀ alkyls, C₈-C₄₀ alicyclics, phenyls substituted with a C₂-C₄₀ alkyl group, C₂-C₄₀ alkyls substituted with an aryl group, and C₈-C₈₀ complex esters, wherein the R⁵ alkyl group optionally comprises at least one substitutent chosen from hydroxyl, alkoxy, and halo groups.

In at least one embodiment, the at least one associative vinyl monomer is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tri(1′-phenylethyl)phenyl (meth)acrylates, polyethoxylated (meth)acrylates of hydrogenated castor oil, polyethoxylated canola (meth)acrylates, polyethoxylated (meth)acrylates of cholesterol, and mixtures thereof, wherein the polyethoxylated portion of the monomer comprises from 5 to 100, such as from 10 to 80, and for example from 15 to 60 ethylene oxide units.

In another embodiment, the at least one associative vinyl monomer is chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates, and polyethoxylated lauryl (meth)acrylates, wherein the polyethoxylated portion of the monomer comprises from 10 to 80, such as from 15 to 60, and for example from 20 to 40 ethylene oxide units.

In a further embodiment, the at least one associative vinyl monomer is present in an amount ranging from 0.001% to 25% by weight, such as from 0.01% to 15% by weight, and for example from 0.1% to 10% by weight, relative to the total weight of the mixture of monomers.

The at least one semi-hydrophobic surfactant vinyl monomer, optionally present in the mixture of monomers, can moderate the associative properties of the cationic associative polymers which comprise them, thus may produce aqueous gels having a very good texture and very good rheological properties.

For the purpose of the present disclosure, the term “semi-hydrophobic surfactant vinyl monomer” is intended to mean a monomer which has a structure similar to an associative monomer, but has a substantially nonhydrophobic end and thus does not confer an associative property on the polymers.

The associativity property of a polymer is linked to the property, in a given medium, of the molecules of said polymer of associating with one another, or of associating with molecules of a coagent, such as a surfactant coagent, which, within a certain concentration range, is reflected by an increase in the viscosity of the medium.

The at least one semi-hydrophobic surfactant vinyl monomer may be compounds having two parts:

A. an unsaturated end group for allowing addition polymerization with the other monomers of the reaction mixture, and

B. a polyoxyalkylene group for reducing the associations between the hydrophobic groups of the polymer or the hydrophobic groups of the other materials possibly present in the composition comprising the polymer.

The end A providing the vinyl or ethylenic unsaturation for the addition polymerization may be, for example, derived from an α,β-ethylenically unsaturated monocarboxylic or dicarboxylic acid or anhydride, such as a C₃-C₄ monocarboxylic or dicarboxylic acid or an anhydride of this acid. Alternatively, the end A may derive from an allyl ether, a vinyl ether or a urethane which is unsaturated and nonionic.

The polymerizable unsaturated end A may also derive from a C₈-C₃₀ unsaturated fatty acid comprising at least one free carboxyl functional group. This C₈-C₃₀ group is then part of the unsaturated end A and is different from the pendant hydrophobic groups of the associative monomers, which are separated from the unsaturated end of the associative monomer by a hydrophilic spacer group.

The polyoxyalkylene portion B comprises a long-chain polyoxyalkylene segment, which is similar to the hydrophilic portion of the associative monomers. In at least one embodiment, the polyoxyalkylene portions B include the C₂-C₄ polyoxyethylene, polyoxypropylene and polyoxybutylene units comprising from 5 to 250, such as from 10 to 100 oxyalkylene units. When the semi-hydrophobic surfactant vinyl monomer comprises more than one type of oxyalkylene unit, these units may be arranged in random or nonrandom sequences or in blocks.

In at least one embodiment, the at least one semi-hydrophobic surfactant vinyl monomer is chosen from the compounds of formula (IV) and (V):

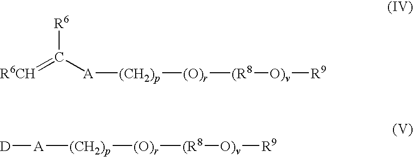

wherein:
each R⁶ independently is chosen from a hydrogen atom, a C₁-C₃₀ alkyl, —C(O)OH, and —C(O)OR⁷;
R⁷ is a C₁-C₃₀ alkyl;
A is chosen from —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, and —CH₂CH₂NHC(O)—;
Ar is a divalent aryl group;
E is chosen from a hydrogen atom and a methyl group;
z is an integer ranging from 0 to 1;
p is an integer ranging from 0 to 30;
r is an integer ranging from 0 to 1, with the provisos that, when p is 0, then r is 0, and when p is an integer ranging from 1 to 30, then r is 1;
(R₈—O)ᵥ is a polyoxyalkylene which is a homopolymer, a random copolymer or a block copolymer comprising C₂-C₄ oxyalkylene units, wherein R⁸ is chosen from C₂H₄, C₃H₆, C₄H₈, and mixtures thereof, and v is an integer ranging from 5 to 250;
R⁹ is chosen from a hydrogen atom and a C₁-C₄ alkyl; and
D is an unsaturated C₈-C₃₀ alkyl optionally substituted with a carboxyl group.

In at least one embodiment, the mixture of monomers comprises at least one semi-hydrophobic surfactant vinyl monomer chosen from one of the following formulae:

CH₂═CH—O(CH₂)ₐO(C₃H₆O)_{b}(C₂H₄O)_{c}H or

CH₂═CHCH₂O(C₃H₆O)_{d}(C₂H₄O)ₑH;

wherein

a is an integer ranging from 2 to 4;

b is an integer ranging from 1 to 10;

c is an integer ranging from 5 to 50;

d is an integer ranging from 1 to 10; and

e is an integer ranging from 5 to 50.

Non-limiting examples of the at least one semi-hydrophobic surfactant vinyl monomer include the polymerizable emulsifiers sold under the references EMULSOGEN® R109, R208, R307, RAL109, RAL208 and RAL307 by the company Clariant; BX-AA-E5P5 sold by the company Bimax; and MAXEMUL® 5010 and 5011 sold by the company Uniqema. In at least one embodiment, the at least one semi-hydrophobic surfactant vinyl monomer is chosen from EMULSOGEN® R208, R307 and RAL307.

According to the manufacturers:

EMULSOGEN® R109 is a random ethoxylated/propoxylated 1,4-butanediol vinyl ether having the empirical formula:

CH₂═CH—O(CH₂)₄O(C₃H₆O)₄(C₂H₄O)₁₀H;

EMULSOGEN® R208 is a random ethoxylated/propoxylated 1,4-butanediol vinyl ether having the empirical formula:

CH₂═CH—O(CH₂)₄O(C₃H₆O)₄(C₂H₄O)₂₀H;

EMULSOGEN® R307 is a random ethoxylated/propoxylated 1,4-butanediol vinyl ether having the empirical formula:

CH₂═CH—O(CH₂)₄O(C₃H₆O)₄(C₂H₄O)₃₀H;

EMULSOGEN® RAL 109 is a random ethoxylated/propoxylated allyl ether having the empirical formula:

CH₂═CHCH₂—O(C₃H₆O)₄(C₂H₄O)₁₀H;

EMULSOGEN® RAL 208 is a random ethoxylated/propoxylated allyl ether having the empirical formula:

CH₂═CHCH₂—O(C₃H₆O)₄(C₂H₄O)₂₀H;

EMULSOGEN® RAL 307 is a random ethoxylated/propoxylated allyl ether having the empirical formula:

CH₂═CHCH₂—O(C₃H₆O)₄(C₂H₄O)₃₀H;

MAXEMUL® 5010 is a hydrophobic carboxylated C₁₂-C₁₅ alkenyl ethoxylated comprising 24 ethylene oxide units;
MAXEMUL® 5011 is a hydrophobic carboxylated C₁₂-C₁₅ alkenyl ethoxylated comprising 34 ethylene oxide units;
and BX-AA-E5P5 is a random ethoxylated/propoxylated allyl ether having the empirical formula:

CH₂═CHCH₂—O(C₃H₆O)₅(C₂H₄O)₅H.

The amount of the at least one semi-hydrophobic surfactant vinyl monomer used in the preparation of the at least one cationic polymer (i) used in the composition according to the disclosure may vary to a large extent and depends, among other things, on the final rheological properties desired for the polymer.

When present, the at least one semi-hydrophobic surfactant vinyl monomer is present in an amount ranging from 0.01% to 25% by weight, and for example from 0.1% to 10% by weight, relative to the total weight of the mixture of monomers.

The at least one cationic polymer (i) used in the composition according to the disclosure is prepared from a mixture of monomers that may comprise at least one hydroxylated nonionic vinyl monomer.

These monomers are monomers comprising at least one ethylenic unsaturation and comprising at least one hydroxyl substituent.

With respect to the at least one hydroxylated nonionic vinyl monomer, non-limiting mention may be made of hydroxylated C₁-C₆ alkyl (meth)acrylates, for example hydroxylated C₁-C₄ alkyl (meth)acrylates, such as 2-hydroxyethyl methacrylate (HEMA), 2-hydroxyethyl acrylate (2-HEA) or 3-hydroxypropyl acrylate; hydroxylated C₁-C₄ alkyl (meth)acrylamides, such as N-(2-hydroxyethyl)methacrylamide, N-(2-hydroxyethyl)acrylamide, N-(3-hydroxypropyl)acrylamide or N-(2,3-dihydroxypropyl)acrylamide; and mixtures thereof. Non-limiting mention may also be made of allyl alcohol, glycerol monoallyl ether, 3-methyl-3-buten-1-ol, vinyl alcohol precursors and equivalents thereof, such as vinyl acetate.

The at least one hydroxylated nonionic vinyl monomer may be present in an amount ranging from 0% to 10% by weight, relative to the total weight of the mixture of monomers. In at least one embodiment, the at least one hydroxylated nonionic vinyl monomer is present in an amount ranging from 0.01% to 10% by weight, such as from 1% to 8%, and for example from 1% to 5% by weight, relative to the total weight of the mixture of monomers.

The at least one cationic polymer (i) in the composition according to the disclosure is prepared from a mixture of monomers which may comprise at least one crosslinking monomer for introducing branches or for controlling the molecular mass.

Polyunsaturated crosslinking agents that can be used are well known in the art. Monounsaturated compounds having a reactive group capable of crosslinking a copolymer formed, before, during or after the polymerization, can also be used. Other non-limiting examples of crosslinking monomers that can be used include polyfunctional monomers comprising multiple reactive groups such as epoxide or isocyanate groups and hydrolysable silane groups. Numerous polyunsaturated compounds may be used to generate a partially or substantially crosslinked three-dimensional network.

Non-limiting examples of polyunsaturated crosslinking monomers that can be used include polyunsaturated aromatic monomers, such as divinylbenzene, divinyl naphthylene and trivinylbenzene; polyunsaturated alicyclic monomers, such as 1,2,4-trivinylcyclohexane; difunctional phthalic acid esters, such as diallyl phthalate; polyunsaturated aliphatic monomers, such as dienes, trienes and tetraenes, for example isoprene, butadiene, 1,5-hexadiene, 1,5,9-decatriene, 1,9-decadiene and 1,5-heptadiene.

Other non-limiting examples of polyunsaturated crosslinking monomers that can be used include polyalkenyl ethers, such as triallyl pentaerythritol, diallyl pentaerythritol, diallyl sucrose, octaallyl sucrose and trimethylolpropane diallyl ether; polyunsaturated esters of polyalcohols or of polyacids, such as 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate and polyethylene glycol di(meth)acrylate; alkylenebisacrylamides, such as methylenebisacrylamide or propylenebisacrylamide; hydroxylated and carboxylated derivatives of methylenebisacrylamide, such as N,N′-bismethylolmethylenebisacrylamide; polyethylene glycol di(meth)acrylates, such as ethylene glycol di(meth)acrylate, diethylene glycol di(meth)acrylate, triethylene glycol di(meth)acrylate, polyunsaturated silanes such as dimethyldivinylsilane, methyltrivinylsilane, allyldimethylvinylsilane, diallyldimethylsilane and tetravinylsilane, and polyunsaturated stannanes, such as tetraallyl tin and diallyldimethyl tin.

Monounsaturated crosslinking monomers that can be used and that bear a reactive group include, for example, N-methylolacrylamides; N-alkoxy(meth)acrylamides, where the alkoxy group is a C₁-C₁₈ group; and unsaturated hydrolysable silanes such as triethoxyvinylsilane, trisisopropoxyvinylsilane and 3-triethoxysilylpropyl methacrylate.

Polyfunctional crosslinking monomers that can be used and that comprise several reactive groups include, for example, hydrolysable silanes such as ethyltriethoxysilane and ethyltrimethoxysilane; hydrolysable epoxy silanes, such as 2-(3,4-epoxycyclohexyl)ethyl triethoxysilane and 3-glycidoxypropyltrimethoxysilane; polyisocyanates, such as 1,4-diisocyanatobutane, 1,6-diisocyanatohexane, 1,4-phenylene diisocyanate and 4,4′-oxybis(phenyl isocyanate); unsaturated epoxides, such as glycidyl methacrylate and allylglycidyl ether; polyepoxides, such as diglycidyl ether, 1,2,5,6-diepoxyhexane and ethylene glycol diglycidyl ether.

Polyunsaturated crosslinking monomers that can be used include, for example, ethoxylated polyols, such as diols, triols and bisphenols, ethoxylated with from 2 to 100 mol of ethylene oxide per mole of hydroxyl functional group and ending with a polymerizable unsaturated group such as a vinyl ether, an allyl ether, an acrylate ester or a methacrylate ester. Such crosslinking monomers may, for example, be ethoxylated bisphenol A dimethacrylate, ethoxylated bisphenol F dimethacrylate, and ethoxylated trimethylolpropane trimethacrylate.

Other non-limiting examples of ethoxylated crosslinking monomers that can be used in the present disclosure include the ethoxylated polyol-derived crosslinking agents disclosed in U.S. Pat. No. 6,140,435.

Further non-limiting examples of crosslinking monomers are polyol acrylate and methacrylate esters comprising at least two acrylate or methacrylate ester groups, such as trimethylolpropane triacrylate (TMPTA), trimethylolpropane dimethacrylate, triethylene glycol dimethacrylate (TEGDMA) and ethoxylated (30) bisphenol A dimethacrylate (EOBDMA).

The at least one crosslinking monomer may be present in an amount ranging from 0% to 5% by weight, relative to the total weight of the mixture of monomers. According to at least one embodiment, the at least one crosslinking monomer is present in an amount ranging from 0.001% to 5% by weight, such as from 0.05% to 2% by weight, and for example from 0.1% to 1% by weight, relative to the total weight of the mixture of monomers.

The mixture of monomers may comprise at least one chain-transfer agent. Chain-transfer agents are compounds well known in the art.

Non-limiting mention may be made of thiolated compounds, disulphide compounds, such as C₁-C₁₈ mercaptans, mercaptocarboxylic acids, mercaptocarboxylic acid esters, thioesters, C₁-C₁₈ alkyl disulphides, aryl disulphides, polyfunctional thiols; phosphites and hypophosphites; haloalkyl compounds, such as carbon tetrachloride, bromotrichloromethane; and unsaturated chain-transfer agents, such as alpha-methylstyrene.

The polyfunctional thiols are, for example, trifunctional thiols, such as trimethylolpropane tris(3-mercaptopropionate), tetrafunctional thiols, such as pentaerythritol tetra(3-mercaptopropionate), pentaerythritol tetra(thioglycolate) and pentaerythritol tetra(thiolactate); and hexafunctional thiols, such as pentaerythritol hexa(thioglyconate).

In another embodiment, the at least one chain-transfer agent may be at least one catalytic chain-transfer agent which reduces the molecular weight of the addition polymers during the free-radical polymerization of the vinyl monomers. Non-limiting mention may be made of cobalt complexes, such as cobalt (II) chelates. The at least one catalytic chain-transfer agent can often be used at low concentrations relative to the thiolated chain-transfer agents.

Non-limiting mention may be made of chain-transfer agents such as octyl mercaptan, n-dodecyl mercaptan, t-dodecyl mercaptan, hexadecyl mercaptan, octadecyl mercaptan (ODM), isooctyl 3-mercaptopropionate (IMP), butyl 3-mercaptopropionate, 3-mercaptopropionic acid, butyl thioglycolate, isooctyl thioglycolate, and dodecyl thioglycolate.

The at least one chain-transfer agent may be present in an amount ranging from 0% to 10% by weight, relative to the total weight of the mixture of monomers. In at least one embodiment, the at least one chain-transfer agent is present in an amount ranging from 0.1% to 5% by weight, relative to the total weight of the mixture of monomers.

The mixture of monomers for preparing the at least one cationic polymer (i) used in the composition according to the disclosure may comprise at least one polymeric stabilizer for obtaining stable dispersions or emulsions. In at least one embodiment, the polymers are water-soluble. Non-limiting mention may be made of synthetic polymers, such as polyvinyl alcohols, partially hydrolysed polyvinyl acetates, polyvinylpyrrolidone, polyacrylamides, polymethacrylamides, carboxylated addition polymers, poly(alkyl vinyl ether)s; water-soluble natural polymers, such as gelatine, peptins, alginates, casein; and modified natural polymers, such as methylcellulose, hydroxypropylcellulose, carboxy-methylcellulose or allyl hydroxyethylcelluloses.

The at least one polymeric stabilizer may be present in an amount ranging from 0% to 2% by weight, relative to the total weight of the mixture of monomers, such as in an amount ranging from 0.0001% to 1% by weight, and for example from 0.01% to 0.5% by weight, relative to the total weight of the mixture of monomers.

According to at least one embodiment, the mixture of monomers comprises, relative to the total weight of the mixture of monomers:
a) from 10% to 70% by weight of the at least one vinyl monomer substituted with at least one amino group,
b) from 20% to 80% by weight of the at least one hydrophobic nonionic vinyl monomer,
c) from 0.001% to 25% by weight of the at least one associative vinyl monomer,
d) from 0% to 25% by weight of the at least one semi-hydrophobic surfactant vinyl monomer,
e) from 0% to 10% by weight of at least one hydroxylated nonionic vinyl monomer,
f) from 0% to 5% by weight of at least one crosslinking monomer,
g) from 0% to 10% by weight of at least one chain-transfer agent, and
h) from 0% to 2% by weight of at least one polymeric stabilizer.

In yet another embodiment, the mixture of monomers comprises, relative to the total weight of the mixture of monomers:
a) from 20% to 60% by weight of the at least one vinyl monomer substituted with at least one amino group,
b) from 20% to 70% by weight of the at least one hydrophobic nonionic vinyl monomer,
c) from 0.01% to 15% by weight of the at least one associative vinyl monomer,
d) from 0.1% to 10% by weight of the at least one semi-hydrophobic surfactant vinyl monomer,
e) from 0.01% to 10% by weight of the at least one hydroxylated nonionic vinyl monomer,
f) from 0.001% to 5% by weight of the at least one crosslinking monomer,
g) from 0.001% to 10% by weight of the at least one chain-transfer agent, and
h) from 0% to 2% by weight of the at least one polymeric stabilizer.

According to another embodiment, the mixture of monomers for preparing the at least one cationic polymer (i) used in the composition according to the disclosure comprises, relative to the total weight of the mixture of monomers:

a) from 20% to 50% by weight of at least one vinyl monomer substituted with at least one amino group chosen from:
3-(N,N-dimethylamino)propyl (meth)acrylate,
N′-(3-N,N-dimethylamino)propyl (meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate,

b) from 50% to 65% by weight of at least one hydrophobic nonionic vinyl monomer chosen from acrylic acid C₁-C₃₀ alkyl esters, methacrylic acid C₁-C₃₀ alkyl esters, and mixtures thereof,

c) from 0.1% to 10% by weight of at least one associative vinyl monomer chosen from polyethoxylated cetyl (meth)acrylates, polyethoxylated cetearyl methacrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-(1′-phenylethyl)phenyl (meth)acrylates, polyethoxylated (meth)acrylates of hydrogenated castor oil, polyethoxylated canola (meth)acrylates, polyethoxylated (meth)acrylates of cholesterol, and mixtures thereof,

d) from 0.1% to 10% by weight of at least one semi-hydrophobic surfactant vinyl monomer chosen from one of the following formulae:

CH₂═CH—O(CH₂)ₐO(C₃H₆O)_{b}(C₂H₄O)_{c}H or

CH₂═CHCH₂O(C₃H₆O)_{d}(C₂H₄O)ₑH;

wherein

a is an integer ranging from 2 to 4;

b is an integer ranging from 1 to 10;

c is an integer ranging from 5 to 50;

d is an integer ranging from 1 to 10; and

e is an integer ranging from 5 to 50,

e) from 0% to 10% by weight of at least one hydroxylated nonionic vinyl monomer,

f) from 0% to 5% by weight of at least one crosslinking monomer,

g) from 0% to 10% by weight of at least one chain-transfer agent, and

h) from 0% to 2% by weight of at least one polymeric stabilizer.

In at least one embodiment, the at least one cationic polymer (i) according to the disclosure is chosen from polymers derived from the polymerization of the mixture of monomers below:

a di(C₁-C₄ alkyl)amino(C₁-C₆ alkyl)methacrylate,

at least one (meth)acrylic acid C₁-C₃₀ alkyl esters,

a polyethoxylated C₁₀-C₃₀ alkyl methacrylate comprising from 20 to 30 mol of ethylene oxide,

a polyethylene glycol/polypropylene glycol 30/5 alkyl ether,

a hydroxy(C₂-C₆ alkyl)methacrylate, and

an ethylene glycol dimethacrylate.

Among the at least one cationic polymer (i) used in the composition according to the disclosure, non-limiting mention may be made of the compound sold by the company Noveon under the name CARBOPOL® Aqua CC Polymer and which corresponds to the INCI name Polyacrylate-1 Crosspolymer.

The Polyacrylate-1 Crosspolymer is the product of the polymerization of a mixture of monomers comprising:

a di(C₁-C₄ alkyl)amino(C₁-C₆ alkyl)methacrylate,

at least one (meth)acrylic acid C₁-C₃₀ alkyl ester,

a polyethoxylated C₁₀-C₃₀ alkyl methacrylate (20-25 mol of ethylene oxide unit),

a polyethylene glycol/polypropylene glycol 30/5 alkyl ether,

a hydroxyl(C₂-C₆ alkyl)methacrylate, and

an ethylene glycol dimethacrylate.

The at least one cationic polymer (i) used in the compositions according to the disclosure may be present in an amount ranging from 0.01% to 10% by weight, such as from 0.05% to 5% by weight, and for example from 0.1% to 1% by weight, relative to the total weight of the composition.

The at least one cationic polymer (i) used in the composition according to the disclosure may be prepared by conventional polymerization techniques, such as emulsion polymerization, as is well known in the polymer field. The polymerization may be carried out by a simple batch process or by a controlled addition process, or the reaction can be initiated in a small reactor and then the bulk of the monomers can be added to the reactor in a controlled manner (seeding process). For example, the polymerization may be carried out at a reaction temperature ranging from 20 to 80° C., even though higher or lower temperatures may be used. To facilitate the emulsification of the mixture of monomers, the emulsion polymerization is carried out in the presence of a surfactant, present in an amount ranging from 1% to 10% by weight, such as from 3% to 8% by weight, and for example from 5% to 7% by weight, relative to the total weight of the emulsion. The emulsion polymerization reaction medium also comprises at least one radical initiator, which may be present, for example, in an amount ranging from 0.01% to 3% by weight, relative to the total weight of the mixture of monomers. The polymerization can be carried out in an aqueous medium or in an aqueous-alcoholic medium at a neutral or weakly alkaline pH.

In a typical polymerization, the mixture of monomers is added with stirring to a solution of emulsifying surfactants, such as a nonionic surfactant, for example a linear or branched alcohol ethoxylate, or a mixture of nonionic and anionic surfactants, such as fatty alcohol sulphates or fatty alcohol alkyl sulphonates, in a suitable amount of water, in a suitable reactor, for preparing the monomer emulsion. The emulsion is deoxygenated by means of any known method, and the polymerization reaction is then initiated by adding a polymerization catalyst (initiator) such as sodium persulphate, or any other suitable addition polymerization catalyst, as is well known in the polymer field. The reaction is stirred until the polymerization is complete, for example, for a period ranging from 4 hours to 16 hours. The monomer emulsion can be heated to a temperature ranging from 20 to 80° C. before the addition of the initiator, if so desired. The amount of monomers having not reacted can be eliminated by the addition of an additional amount of catalyst. The polymer emulsion obtained can be withdrawn from the reactor and packaged so as to be stored or used. Optionally, the pH or other physical or chemical characteristics of the emulsion can be adjusted before the emulsion is withdrawn from the reactor. The emulsion produced can have a total solids content which ranges from 10% to 40% by weight. The total amount of polymers in the emulsion obtained can range from 15% to 35% by weight, and can be at most 25% by weight.

Surfactants suitable for facilitating the emulsion polymerization may be nonionic, anionic, amphoteric or cationic surfactants, or mixtures thereof. Nonionic or anionic surfactants, or mixtures thereof, are most commonly used.

All types of non ionic, anionic, amphoteric or cationic surfactants conventionally used in emulsion polymerizations may be used.

The polymerization may be carried out in the presence of at least one free-radical initiator. The latter may be chosen from insoluble inorganic persulphate compounds, such as ammonium persulphate, potassium persulphate or sodium persulphate; peroxides such as hydrogen peroxide, benzoyl peroxide, acetyl peroxide and lauryl peroxide; organic hydroperoxides, such as cumen hydroperoxide and t-butyl hydroperoxide; organic peracids such as peracetic acid; and oil-soluble free-radical-producing agents, such as 2,2′-azobisisobutyronitrile, and mixtures thereof. The peroxides and the peracids may be optionally activated with reducing agents, such as sodium bisulphite or ascorbic acid, transition metals or hydrazine. Free-radical initiators which are suitable include, but not limited to, water-soluble azo polymerization initiators, such as 2,2′-azobis(tert-alkyl) compounds having a water-soluble substituent on the alkyl group. Non-limiting examples of azo polymerization catalysts include the free-radical initiators VAZO®, sold by the company DuPont, such as VAZO® 44 (2,2′-azobis(2,4,5-dihydroimidazolyl)propane), VAZO® 56 (2,2′-azobis(2-methylpropionamidine) dihydrochloride), and VAZO® 68 (4,4′-azobis(4-cyanovaleric acid)).

The at least one starch that can be used in the present disclosure includes macromolecules in the form of polymers made from elementary units which are anhydroglucose units. The number of these units and the assembly thereof make it possible to distinguish amylose (linear polymer) and amylopectin (branched polymer). The relative proportions of amylose and of amylopectin, and also the degree of polymerization thereof, vary according to the botanical origin of the starches.

The botanical origin of the molecules of starches used in the present disclosure may be cereals or tubers. Thus, the starches may be, for example, chosen from maize, rice, cassava, barley, potato, wheat, sorghum, and pea starches.

The starches used in the composition of the disclosure are, in at least one embodiment, modified. The starches may be modified chemically or physically, such by at least one of the following reactions: pregelatinization, oxidation, crosslinking, esterification, etherification, amidation, and heat treatments.

These reactions may be carried out in the following way:

pregelatinization, causing the starch granules to rupture (for example, drying and cooking in a drum dryer);

oxidation with strong oxidants, resulting in the introduction of carboxyl groups into the starch molecule and in the depolymerization of the starch molecule (for example, by treating an aqueous solution of starch with sodium hypochlorite);

crosslinking with functional agents capable of reacting with the hydroxyl groups of the starch molecules, which will thus be linked to one another (for example, with glyceryl and/or phosphate groups); and

esterification in an alkaline medium for grafting functional groups, for example C₁-C₆ acyl (such as acetyl), C₁-C₆ hydroxyalkylated (such as hydroxyethyl, hydroxypropyl), carboxymethyl or octenylsuccinic functional groups.

It is possible to obtain, by crosslinking with phosphorus-containing compounds, monostarch phosphates (of the Am—O—PO—(OX)₂ type), distarch phosphates (of the Am—O—PO—(OX)—O—Am type) or even tristarch phosphates (of the Am—O—PO—(O—Am)₂ type) or mixtures thereof.

X may be chosen from alkaline metals (for example, sodium or potassium), alkaline earth metals (for example, calcium or magnesium), aqueous ammonia salts, amine salts such as monoethanolamine salts, diethanolamine salts, triethanolamine salts or 3-amino-1,2-propanediol salts, or ammonium salts derived from basic amino acids such as lysine, arginine, sarcosine, ornithine or citrulline. Am is a starch.

The phosphorus-containing compounds may, for example, be sodium tripolyphosphate, sodium orthophosphate, phosphorus oxychloride or sodium trimetaphosphate.

In at least one embodiment, distarch phosphates or compounds rich in distarch phosphate, such as the product proposed under the references PREJEL VA-70-T AGGL (gelatinized hydroxypropylated cassava distarch phosphate) or PREJEL TK1 (gelatinized cassava distarch phosphate) or PREJEL 200 (gelatinized acetylated cassava distarch phosphate) by the company Avebe, or STRUCTURE® Zea from National Starch (gelatinized maize distarch phosphate) may be used.

In yet another embodiment, the at least one starch is a starch having undergone at least one chemical modification such as at least one esterification.

According to the disclosure, use may also be made of amphoteric starches comprising at least one anionic group and at least one cationic group. The anionic and cationic groups may be linked to the same reactive site of the starch molecule or to different reactive sites; in at least one embodiment, they are linked to the same reactive site. The anionic groups may be of carboxylic, phosphate or sulphate type, for example carboxylic type. The cationic groups may be of primary, secondary, tertiary or quaternary amine type.

The amphoteric starches may be, for example, chosen from the compounds of formulae below:

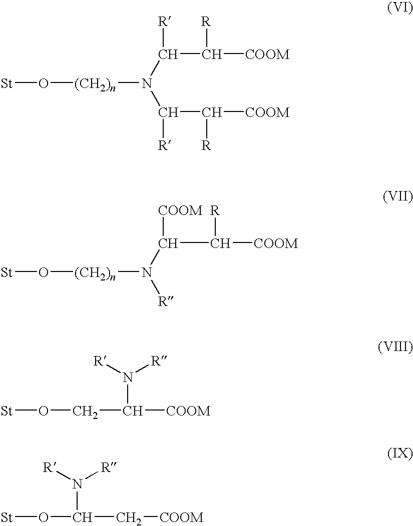

wherein:
St-O is a starch molecule,
R, which may be identical or different, is chosen from a hydrogen atom and a methyl radical,
R′, which may be identical or different, is chosen from a hydrogen atom, a methyl radical, and a —COOH group,
n is an integer ranging from 2 to 3,
M, which may be identical or different, is chosen from a hydrogen atom, an alkaline metal or an alkaline earth metal, such as Na, K or Li, NH₄, a quaternary ammonium, and an organic amine; and
R″ is chosen from a hydrogen atom and an alkyl radical comprising from 1 to 18 carbon atoms.

These compounds are, for example, described in U.S. Pat. No. 5,455,340 and U.S. Pat. No. 4,017,460, which are incorporated herein by reference.

The starch molecules may be derived from all plant sources of starch, such as corn, potato, oats, rice, tapioca, sorghum, barley or wheat. Hydrolysates of the starches mentioned above may also be used. The starch is, in at least one embodiment, derived from potato.

In at least one further embodiment, the starches of formula (VI) or (VII) are used. Starches modified with 2-chloroethylaminodipropionic acid, for example the starches of formula (VI) or (VII) wherein R, R′, R″ and M are hydrogen atoms and n is equal to 2, are used in yet another embodiment. In at least one embodiment, the amphoteric starch is chosen from a starch chloroethylamidodipropionate.

According to the disclosure, the at least one starch may be present in an amount ranging from 0.1% to 20% by weight, such as from 0.5% to 15% by weight, and for example from 1% to 10% by weight, relative to the total weight of the final composition.

In at least one embodiment, the ratio by weight of the at least one cationic polymer (i) described above to the at least one starch ranges from 0.01 to 10, and for example from 0.05 to 5, such as from 0.1 to 1.

The compositions according to the disclosure, in at least one embodiment, also comprise at least one surfactant, such as those chosen from anionic, amphoteric, nonionic and cationic surfactants, and mixtures thereof.

Among the anionic surfactants, non-limiting mention may be made of alkaline metal salts, ammonium salts, amine salts, amino alcohol salts and magnesium salts of the following compounds: alkyl sulphates, alkyl ether sulphates, alkylamido ether sulphates, alkylarylpolyether sulphates, monoglyceride sulphates; alkyl sulphonates, alkylamide sulphates, alkylaryl sulphonates, olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl phosphates, alkyl ether phosphates; and acylsarcosinates, acylisethionates and N-acyltaurates.

The alkyl or acyl radical of these various compounds may comprise a carbon-based chain comprising from 8 to 30 carbon atoms.

Among the anionic surfactants, non-limiting mention may also be made of fatty acid salts such as oleic acid, ricinoleic acid, palmitic acid or stearic acid salts; coconut oil or hydrogenated coconut oil acids; and acyl lactylates, the acyl radical of which comprises from 8 to 30 carbon atoms.

Use may also be made of surfactants considered to be weakly anionic, such as polyoxyalkylenated carboxylic alkyl or alkylaryl ether acids or salts thereof, polyoxyalkylenated carboxylic alkylamido ether acids or salts thereof, and alkyl D-galactosiduronic acids or salts thereof.

The nonionic surfactants are, in at least one embodiment, chosen from polyethoxylated, polypropoxylated or polyglycerolated fatty acids or alkylphenols or alcohols, with a fatty chain comprising from 8 to 30 carbon atoms, the number of ethylene oxide or propylene oxide groups comprising from 2 to 50 and the number of glycerol groups comprising from 2 to 30.

Non-limiting mention may also be made of copolymers of ethylene oxide and of propylene oxide; condensates of ethylene oxide and of propylene oxide with fatty alcohols; polyethoxylated fatty amides, for example comprising from 2 to 30 mol of ethylene oxide; polyglycerolated fatty amides, for example comprising from 1 to 5 glycerol groups, such as from 1.5 to 4; polyethoxylated fatty amines, for example comprising from 2 to 30 mol of ethylene oxide; oxyethylenated sorbitan fatty acid esters comprising from 2 to 30 mol of ethylene oxide; sucrose fatty acid esters, polyethylene glycol fatty acid esters, alkylpolyglycosides, carbamate or amide derivatives of N-alkylglucamines, aldobionamides, and amine oxides such as alkylamine oxides or N-acylamidopropylmorpholine oxides.

In at least one embodiment, the amphoteric surfactants are chosen from secondary or tertiary aliphatic amine derivatives, wherein the aliphatic radical is a linear or branched chain comprising from 8 to 22 carbon atoms and comprising at least one carboxylate, sulphonate, sulphate, phosphate or phosphonate water-solubilizing anionic group; (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives, non-limiting mention may be made of the products sold under the name MIRANOL, as described in U.S. Pat. No. 2,528,378 and U.S. Pat. No. 2,781,354 and classified in the CTFA dictionary, 7th edition, 1997, under the name Disodium Cocoamphodiacetate, Disodium Lauroamphodiacetate, Disodium Capryloamphodiacetate, Disodium Caproamphodiacetate, Disodium Cocoamphodipropionate, Disodium Lauroamphodipropionate, Disodium Caproamphodipropionate, Disodium Capryloamphodipropionate, Lauroamphodipropionate acid, and Cocoamphodipropionate acid.

The cationic surfactants are, in at least one embodiment, chosen from optionally polyoxyalkylenated primary, secondary or tertiary fatty amine salts; quaternary ammonium salts; imidazoline derivatives; or amine oxides of cationic nature.

Non-limiting examples of the quaternary ammonium salts include tetraalkylammonium halides (for example chlorides) such as dialkyldimethylammonium or alkyltrimethylammonium chlorides, wherein the alkyl radical comprises from approximately 12 to 22 carbon atoms, for example behenyltrimethylammonium, distearyldimethylammonium, cetyltrimethylammonium or benzyldimethylstearylammonium chloride or alternatively stearamidopropyldimethyl(myristyl acetate)ammonium chloride sold under the name “CEPHARYL 70” by the company Van Dyk.

Diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (such as chlorides or methylsulphate) and mixtures thereof may also be used. The acyl radicals may comprise from 14 to 18 carbon atoms and for example originate from a plant oil such as palm oil or sunflower oil.

The surfactants are optionally present in the compositions in accordance with the disclosure in an amount ranging from 0.01% to 50% by weight, relative to the total weight of the composition. When the compositions are in the form of shampoos, they may be present in an amount of at least 4% by weight, such as ranging from 5% to 50% by weight, relative to the total weight of the composition, and for example from 8% to 35%.

The compositions according to the disclosure may have a pH ranging from 3 to 12, and for example from 4 to 8, such as from 4 to 6.

According to at least one embodiment, the cosmetic compositions may also comprise at least one conditioning agent for keratin materials.

When the composition comprises at least one conditioning agent, the at least one conditioning agent may be chosen from synthetic oils such as poly-α-olefins, fluoro oils, fluoro waxes, fluoro gums, water-insoluble carboxylic acid esters, cationic polymers other than the at least one cationic polymer (i) of the disclosure, silicones, mineral, plant or animal oils, ceramides and pseudoceramides, and mixtures thereof.

The polyolefins may be poly-α-olefins, such as:

hydrogenated or nonhydrogenated polybutene type, and for example hydrogenated or nonhydrogenated polyisobutene type.

Use can be made of oligomers of isobutylene with a molecular weight of less than 1,000, and mixtures thereof with polyisobutylenes with a molecular weight of greater than 1,000, and for example ranging from 1,000 to 15,000.

Non-limiting examples of poly-α-olefins that can be used in the context of the present disclosure include the polyisobutenes sold under the name PERMETHYL® 99 A, 101 A, 102 A, 104 A (n=16) and 106 A (n=38) by the company Presperse Inc., or the products sold under the name ARLAMOL HD (n=3) by the company ICI (n is the degree of polymerization);

hydrogenated or nonhydrogenated polydecene type.

Such products are sold, for example, under the names ETHYLFLO by the company Ethyl Corp., and ARLAMOL PAO by the company ICI.

The mineral oils that can be used in the compositions of the disclosure may be chosen from the group made up of:

hydrocarbons, such as hexadecane and liquid paraffin.

In another embodiment, the compositions of the disclosure also comprise at least one cationic polymer different from the at least one cationic polymer (i) of the disclosure chosen from all those already known, such as those described in Patent Application EP-A-0 337 354 and in French Patent Applications FR-A-2 270 846, 2 383 660, 2 598 611, 2 470 596 and 2 519 863.

The at least one cationic polymer different from the at least one cationic polymer (i) of the disclosure used may have a molecular weight ranging from 500 to 5×10⁶, and for example from 10³ to 3×10⁶.

Among the at least one cationic polymer different from the at least one cationic polymer (i) of the disclosure, non-limiting mention may be made of quaternized proteins (or protein hydrolysates) and polymers of the polyamine, polyaminoamide and polyquaternary ammonium type. They are known products.

The quaternized protein or protein hydrolysates may be chemically modified polypeptides bearing quaternary ammonium groups at the end of the chain or grafted onto said chain. Their molecular weight can range, for example, from 1,500 to 10,000, and for example from 2,000 to 5,000. Among these compounds, non-limiting mention may be made of:

hydrolysates of collagen bearing triethylammonium groups, such as the products called, in the CTFA dictionary, “Triethonium Hydrolyzed Collagen Ethosulphate”;

hydrolysates of collagen bearing trimethylammonium chloride and trimethylstearylammonium chloride groups, called, in the CTFA dictionary, “Steartrimonium Hydrolyzed Collagen”;

hydrolysates of proteins bearing, on the polypeptide chain, quaternary ammonium groups comprising at least one alkyl radical comprising from 1 to 18 carbon atoms.

Among these protein hydrolysates, non-limiting mention may be made, inter alia, of “CROQUAT L”, “CROQUAT M”, “CROQUAT S” and “CROTEIN Q” sold by the company Croda.

Other quaternized proteins or hydrolysates are, for example, those sold by the company Inolex under the name “LEXEIN QX 3000”.

Non-limiting mention may also be made of quaternized plant proteins, such as wheat, corn or soybean proteins; as quaternized wheat proteins, non-limiting mention may be made of those called, in the CTFA dictionary, “Cocodimonium Hydrolysed Wheat Protein”, “Lauridimonium Hydrolysed Wheat Protein”, or “Steardimonium Hydrolysed Wheat Protein”.

The polymers of the polyamine, polyamidoamide or polyquaternary ammonium type that can be used in accordance with the present disclosure include those described in French Patents 2 505 348 or 2 542 997. Among these polymers, non-limiting mention may be made of:

(1) Homopolymers or copolymers derived from acrylic or methacrylic esters or amides and comprising at least one of the units of the following formulae:

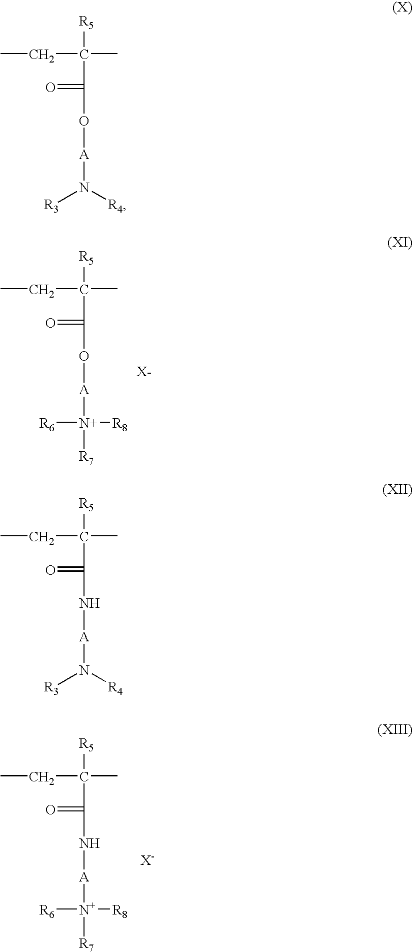

wherein:
R₃ and R₄, which may be identical or different, are chosen from hydrogen atoms and alkyl groups comprising from 1 to 6 carbon atoms, such as methyl or ethyl;
R₅, which may be identical or different, is chosen from a hydrogen atom and a CH₃ radical;
A, which may be identical or different, is chosen from a linear or branched alkyl group comprising from 1 to 6 carbon atoms, such as 2 or 3 carbon atoms, and a hydroxyalkyl group comprising from 1 to 4 carbon atoms;
R₆, R₇ and R₈, which may be identical or different, are chosen from alkyl groups comprising from 1 to 18 carbon atoms and benzyl radicals, for example an alkyl group comprising from 1 to 6 carbon atoms;
X is an anion derived from a mineral or organic acid, such as a methosulphate anion or a halide such as chloride or bromide.

The copolymers of family (1) may also comprise at least one unit derived from comonomers that may be chosen from the family of acrylamides, methacrylamides, diacetone acrylamides, acrylamides and methacrylamides substituted on the nitrogen with (C₁-C₄) lower alkyls, acrylic or methacrylic acids or esters thereof, vinyllactams such as vinylpyrrolidone or vinylcaprolactam, and vinyl esters.

Thus, among these copolymers of family (1), non-limiting mention may be made of:

copolymers of acrylamide and of dimethylaminoethyl methacrylate quaternized with dimethyl sulphate or with a dimethyl halide, such as that sold under the name HERCOFLOC by the company Hercules,

copolymers of acrylamide and of methacryloyloxyethyltrimethylammonium chloride described, for example, in Patent Application EP-A-080 976 and sold under the name BINAQUAT P 100 by the company Ciba Geigy,

the copolymers of acrylamide and of methacryloyloxyethyltrimethylammonium methosulphate sold under the name RETEN by the company Hercules,

quaternized or nonquaternized vinylpyrrolidone/dialkylaminoalkyl acrylate or methacrylate copolymers, such as the product sold under the name “GAFQUAT®” by the company ISP, for instance “GAFQUAT® 734” or “GAFQUAT® 755” or the products known as “COPOLYMER 845, 958 and 937”. These polymers are described in detail in French Patents 2 077 143 and 2 393 573,

dimethylaminoethyl methacrylate/vinylcaprolactam/vinylpyrrolidone terpolymers, such as the product sold under the name GAFFIX® VC 713 by the company ISP,

vinylpyrrolidone/methacrylamidopropyldimethylamine copolymers sold, for example, under the name STYLEZE® CC 10 by ISP,

and quaternized vinylpyrrolidone/dimethylaminopropylmethacrylamide copolymers, such as the product sold under the name “GAFQUAT® HS 100” by the company ISP.

Crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, such as methylenebisacrylamide. Use may be made, for example, of a crosslinked acrylamide/methacryloyloxyethyltrimethylammonium chloride copolymer (20/80 by weight) in the form of a dispersion comprising 50% by weight of said copolymer in mineral oil. This dispersion is sold under the name “SALCARE® SC 92” by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names “SALCARE® SC 95” and “SALCARE® SC 96” by the company Ciba;

(2) polymers constituted of piperazinyl units and of divalent alkylene or hydroxyalkylene radicals having straight or branched chains, optionally interrupted with oxygen, sulphur or nitrogen atoms or with aromatic or heterocyclic rings, and also the products of oxidation and/or quaternization of these polymers. Such polymers are, for example, described in French Patents 2 162 025 and 2 280 361;

(3) water-soluble polyaminoamides prepared for example by polycondensation of an acid compound with a polyamine; these polyaminoamides may be crosslinked with an epihalohydrin, a diepoxide, a dianhydride, an unsaturated dianhydride, a bisunsaturated derivative, a bishalohydrin, a bisazetidinium, a bishaloacyldiamine or a bis(alkyl halide), or with an oligomer resulting from the reaction of a bifunctional compound which is reactive with respect to a bishalohydrin, a bisazetidinium, a bishaloacyldiamine, a bis(alkyl halide), an epilhalohydrin, a diepoxide or a bisunsaturated derivative; the crosslinking agent may be used in proportions ranging from 0.025 to 0.35 mol per amine group of the polyaminoamide; these polyaminoamides may be alkoylated or, if they comprise at least one tertiary amine function, quaternized. Such polymers are, for example, described in French Patents 2 252 840 and 2 368 508;

(4) polyaminoamide derivatives resulting from the condensation of polyalkylenepolyamines with polycarboxylic acids, followed by alkylation with bifunctional agents. Non-limiting mention may be made of adipic acid/dialkylaminohydroxyalkyldialkylenetriamine polymers wherein the alkyl radical comprises from 1 to 4 carbon atoms and for example is chosen from methyl, ethyl, and propyl. Such polymers are, for example, described in French Patent 1 583 363.

Among these derivatives, non-limiting mention may be made of the adipic acid/dimethylaminohydroxypropyldiethylenetriamine polymers sold under the name “CARTARETIN F, F4 or F8” by the company Sandoz;

(5) polymers obtained by reaction of a polyalkylenepolyamine comprising two primary amine groups and at least one secondary amine group with a dicarboxylic acid chosen from diglycolic acid and saturated aliphatic dicarboxylic acids comprising from 3 to 8 carbon atoms. The molar ratio of the polyalkylenepolyamine to the dicarboxylic acid is in a range from 0.8:1 to 1.4:1; the polyaminoamide resulting from this reaction being reacted with epichlorohydrin in a molar ratio of epichlorohydrin relative to the secondary amine group of the polyaminoamide in a range from 0.5:1 to 1.8:1. Such polymers are, for example, described in U.S. Pat. Nos. 3,227,615 and 2,961,347.

Polymers of this type are, for example, sold under the name “HERCOSETT 57” by the company Hercules Inc. or under the name “PD 170” or “DELSETTE 101” by the company Hercules in the case of the adipic acid/epoxypropyldiethylenetriamine copolymer;

(6) alkyldiallylamine or dialkyldiallylammonium cyclopolymers, such as homopolymers or copolymers comprising, as main constituent of the chain, units corresponding to formula (XIV) or (XV):

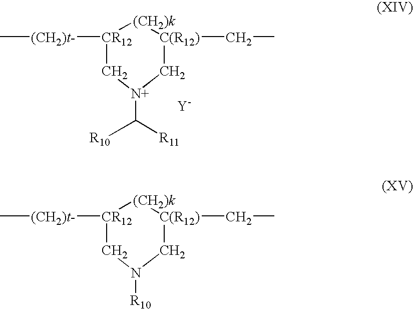

wherein k and t are integers ranging from 0 to 1, the sum k+t being equal to 1; R₁₂ is chosen from a hydrogen atom and a methyl radical; R₁₀ and R₁₁, which may be identical or different, are chosen from alkyl groups comprising from 1 to 6 carbon atoms, hydroxyalkyl groups wherein the alkyl group comprising, for example, from 1 to 5 carbon atoms, and (C₁-C₄) lower amidoalkyl groups, or R₁₀ and R₁₁, together with the nitrogen atom to which they are attached, are chosen from heterocyclic groups, such as piperidinyl or morpholinyl; and Y⁻ is an anion such as bromide, chloride, acetate, borate, citrate, tartrate, bisulphate, bisulphite, sulphate or phosphate. These polymers are, for example, described in French Patent 2 080 759 and in its certificate of addition 2 190 406.

In at least one embodiment, R₁₀ and R₁₁, which may be identical or different, are chosen from alkyl groups comprising from 1 to 4 carbon atoms.

Among the polymers defined above, non-limiting mention may be made of the dimethyldiallylammonium chloride homopolymer sold under the name “MERQUAT 100” by the company Nalco (and its homologues with low weight-average molecular weights) and the copolymers of diallyidimethylammonium chloride and of acrylamide sold under the name “MERQUAT 550”;

(7) the diquaternary ammonium polymer comprising repeat units corresponding to the formula:

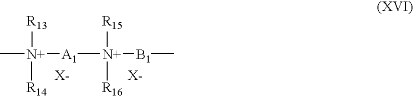

wherein:
R₁₃, R₁₄, R₁₅ and R₁₆, which may be identical or different, are chosen from aliphatic, alicyclic and arylaliphatic radicals comprising from 1 to 20 carbon atoms and lower hydroxyalkylaliphatic radicals, or R₁₃, R₁₄, R₁₅ and R₁₆, together or separately, constitute, with the nitrogen atoms to which they are attached, heterocycles optionally comprising a second heteroatom other than nitrogen, or R₁₃, R₁₄, R₁₅ and R₁₆ are chosen from linear and branched C₁-C₆ alkyl radicals substituted with a nitrile, ester, acyl, amide, —CO—O—R₁₇-D or —CO—NH—R₁₇-D group wherein R₁₇ is an alkylene and D is a quaternary ammonium group;
A₁ and B₁, which may be identical or different, are chosen from polymethylene groups comprising from 2 to 20 carbon atoms that may be linear or branched, saturated or unsaturated, and that may comprise, linked to or intercalated in the main chain, at least one aromatic ring, or at least one oxygen or sulphur atom or sulphoxide, sulphone, disulphide, amino, alkylamino, hydroxyl, quaternary ammonium, ureido, amide or ester groups; and
X⁻ is an anion derived from a mineral or organic acid;
A₁, R₁₃ and R₁₅ can form, with the two nitrogen atoms to which they are attached, a piperazine ring; in addition, if A₁ is a saturated or unsaturated, linear or branched alkylene or hydroxyalkylene radical, B₁ can also be a (CH₂)ₙ—CO-D-OC—(CH₂)ₚ— group wherein p is an integer ranging from 2 to 20; n is an integer ranging from 2 to 20; and D is chosen from:

a) a glycol residue of formula: —O-Z-O—, wherein Z is chosen from a linear or branched hydrocarbon-based radical and a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y are chosen from an integer ranging from 1 to 4, representing a defined and unique degree of polymerization, or any number from 1 to 4, representing a mean degree of polymerization;

b) a bissecondary diamine residue such as a piperazine derivative;

c) a bisprimary diamine residue of the formula: —NH—Y—NH—, wherein Y is chosen from a linear or branched hydrocarbon-based radical and the divalent radical

—CH₂—CH₂—S—S—CH₂—CH₂—; and

d) a ureylene group of the formula: —NH—CO—NH—.

In at least one embodiment, X⁻ is an anion such as chloride or bromide.

These polymers may have a number-average molecular weight ranging from 1,000 to 100,000.

Polymers of this type are, for example, described in French Patents 2 320 330, 2 270 846, 2 316 271, 2 336 434 and 2 413 907, and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

Use may be made, for example, of the polymers which are constituted of repeat units corresponding to the formula:

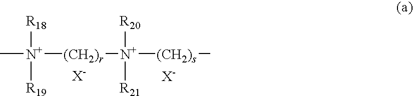

wherein R₁₈, R₁₉, R₂₀ and R₂₁, which may be identical or different, are chosen from alkyl and hydroxyalkyl radicals comprising from 1 to 4 carbon atoms approximately, r and s are integers ranging from 2 to 20 approximately and X— is an anion derived from a mineral or organic acid.

In at least one embodiment, R₁₈, R₁₉, R₂₀ and R₂₁ are methyl radicals, r is 3, s is 6, and X is Cl, known as hexadimethrine chloride according to INCI nomenclature (CTFA);

(8) polyquaternary ammonium polymers constituted of units of formula (XVII):

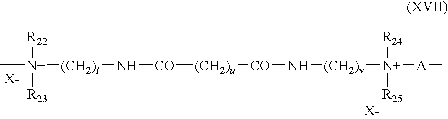

wherein:
R₂₂, R₂₃, R₂₄ and R₂₅, which may be identical or different, are chosen from hydrogen atoms, and methyl, ethyl, propyl, β-hydroxyethyl, β-hydroxypropyl, and —CH₂CH₂(OCH₂CH₂)ₚOH radicals,
p is an integer ranging from 0 to 6, with the proviso that R₂₂, R₂₃, R₂₄ and R₂₅ are not simultaneously hydrogen atoms;
t and u, which may be identical or different, are integers ranging from 1 to 6,
v is an integer ranging from 0 to 34,
X′ is an anion such as a halide,
A is chosen from a radical of a dihalide and —CH₂—CH₂—O—CH₂—CH₂—.

Such compounds are, for example, described in Patent Application EP-A-122 324.

Non-limiting mention may, for example, be made, among these, of the products “MIRAPOL® A 15”, MIRAPOL® AD1”, “MIRAPOL® AZ1” and “MIRAPOL® 175” sold by the company Miranol;

(9) Quaternary polymers of vinylpyrrolidone and of vinylimidazole, such as the products sold under the names LUVIQUAT® FC 905, FC 550 and FC 370 by the company BASF;

(10) Cationic polysaccharides, such as celluloses and gums of cationic galactomannans. Among the cationic polysaccharides, non-limiting mention may be made of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French Patent 1 492 597. These polymers are also defined in the CTFA dictionary as quaternary ammoniums of hydroxyethylcellulose having reacted with an epoxide substituted with a trimethylammonium group.

The cationic cellulose copolymers or the cellulose derivatives grafted with a water-soluble quaternary ammonium monomer are described, for example, in U.S. Pat. No. 4,131,576, such as hydroxyalkylcelluloses, for instance hydroxymethyl-, hydroxyethyl- or hydroxypropylcelluloses grafted, for example, with a methacryloylethyltrimethylammonium, methacrylamidopropyltrimethylammonium or dimethyldiallylammonium salt.

The cationic galactomannan gums are described, for example, in U.S. Pat. Nos. 3,589,578 and 4,031,307, such as guar gums comprising trialkylammonium cationic groups. Guar gums modified with a 2,3-epoxypropyltrimethylammonium salt (for example, chloride) may, for example, be used.

Other cationic polymers that can be used in the context of the disclosure are cationic proteins or cationic protein hydrolysates, polyalkyleneimines, such as polyethyleneimines, polymers comprising vinylpyridine or vinylpyridinium units, condensates of polyamines and of epichlorohydrin, polyquaternary ureylenes and chitin derivatives.

Among all the cationic polymers that can be employed in the context of the present disclosure, non-limiting mention may be made of quaternary cellulose ether derivatives such as the products sold under the name “JR 400” by the company Union Carbide Corporation, cyclopolymers, such as homopolymers of a diallyidimethylammonium salt and copolymers of a diallyldimethylammonium salt and of acrylamide, such as the chlorides, sold under the names “MERQUAT 550” and “MERQUAT S” by the company Merck, cationic polysaccharides, and for example the guar gums modified with 2,3-epoxypropyltrimethylammonium chloride sold, for example, under the name “JAGUAR C13S” by the company Meyhall, optionally crosslinked homopolymers and copolymers of a (meth)acryloyloxyethyltrimethylammonium salt, sold by the company Allied Colloids, in solution at 50% in mineral oil, under the names SALCARE SC92 (crosslinked copolymer of methacryloyloxyethyltrimethylammonium chloride and of acrylamide) and SALCARE SC95 (crosslinked homopolymer of methacryloyloxyethyltrimethylammonium chloride), quaternary copolymers of vinylpyrrolidone and of a vinylimidazole salt, such as the products sold by BASF under the names LUVIQUAT FC 370, LUVIQUAT FC 550, LUVIQUAT FC 905, and LUVIQUAT HM-552.

The additional cationic polymers may be present in the compositions of the disclosure in an amount ranging from 0.001% to 20% by weight, and for example from 0.05% to 5% by weight, such as from 0.1% to 3% by weight, relative to the total weight of the composition.

In at least one embodiment of the disclosure, the compositions according to the present disclosure also comprise at least one silicone, for example modified or unmodified polyorganosiloxanes, such as polyorganosiloxane oils or polyorganosiloxane gums or resins, as they are, or in the form of solutions in organic solvents or in the form of emulsions or microemulsions.

Among the polyorganosiloxanes that can be used in accordance with the present disclosure, non-limiting mention may be made of:

I. Volatile silicones: these have a boiling point ranging from 60° C. to 260° C. They are chosen from cyclic silicones comprising from 3 to 7 silicon atoms, such as from 4 to 5. They include, for example, the octamethylcyclotetrasiloxane sold under the name “VOLATILE SILICONE 7207” by Union Carbide or “SILBIONE 70045 V2” by Rhone Poulenc, the decamethylcyclopentasiloxane sold under the name “VOLATILE SILICONE 7158” by Union Carbide and “SILBIONE 70045 V5” by Rhone Poulenc, and mixtures thereof. Non-limiting mention may also be made of cyclocopolymers of the dimethylsiloxane/methylalkylsiloxane type, such as the “SILICONE VOLATILE FZ 3109” sold by the company Union Carbide, which is a dimethylsiloxane/methyloctylsiloxane cyclocopolymer.

II. Nonvolatile silicones which may comprise:

(i) polyalkylsiloxanes; among the polyalkylsiloxanes, non-limiting mention may be made of linear polydimethylsiloxanes having trimethylsilyl end groups, for instance, and in a nonlimiting manner, the “SILBIONE” oils of the 70047 series sold by Rhodia Chimie; the DC 200 oils from Dow Corning, and polydimethylsiloxanes (PDMSs) having hydroxydimethylsilyl end groups;

(ii) polyarylsiloxanes;

(iii) polyalkylarylsiloxanes; non-limiting mention may be made of linear and branched polymethylphenylsiloxanes, polydimethylmethylphenylsiloxanes and polydimethyldiphenylsiloxanes, such as the oil “RHODORSIL 70763” from Rhodia Chimie;

(iv) silicone gums; these are polydiorganosiloxanes having a molecular weight in a range from 200,000 to 5,000,000, used alone or as a mixture in a solvent chosen from volatile silicones, polydimethylsiloxane (PDMS) oils, polyphenylmethylsiloxane (PPMS) oils, isoparaffins, methylene chloride, pentane, dodecane, tridecane, tetradecane, and mixtures thereof; non-limiting mention is made of the following compounds:

polydimethylsiloxane gums,

poly[(dimethylsiloxane)/(methylvinylsiloxane)] gums

poly[(dimethylsiloxane)/(diphenylsiloxane)] gums,

poly[(dimethylsiloxane)/(phenylmethylsiloxane)] gums, and

poly[(dimethylsiloxane)/(diphenylsiloxane)/(methylvinylsiloxane)] gums.

Non-limiting mention may also be made of the following mixtures:

1) mixtures formed from a polydimethylsiloxane gum hydroxylated at the end of the chain (Dimethiconol according to the CTFA nomenclature), and from a cyclic polydimethylsiloxane (Cyclomethicone according to the CTFA nomenclature), such as the product “Q2 1401” sold by the company Dow Corning;

2) mixtures formed from a polydimethylsiloxane gum with a cyclic silicone, such as the product “SF 1214 SILICONE FLUID” from General Electric, which is an SE 30 gum of molecular weight 500,000 solubilized in “SF 1202 SILICONE FLUID” (decamethylcyclopentasiloxane);

3) mixtures of two PDMSs of different viscosity, for example of a PDMS gum and of a PDMS oil, such as the products “SF 1236” and “CF 1241” from the company General Electric;

(v) silicone resins: for example, crosslinked siloxane systems comprising R₂SiO_{2/2}, RSiO_{3/2} and SiO_{4/2} units wherein R is chosen from a hydrocarbon-based group comprising from 1 to 6 carbon atoms and a phenyl group. Among these resins, non-limiting mention may be made of the product sold under the name “DOW CORNING® 593”;

(vi) organomodified polyorganosiloxanes; i.e., silicones as defined above, comprising, in their general structure at least one organofunctional group directly attached to the siloxane chain or attached by means of a hydrocarbon-based radical; non-limiting mention may be made of silicones comprising:

a) polyethyleneoxy and/or polypropyleneoxy groups, optionally comprising alkyl groups, such as the product known as dimethicone copolyol sold by the company Dow Corning under the name “DC 1248”, and the alkyl (C12) methicone copolyol sold by the company Dow Corning under the name “Q2 5200”;

b) (per)fluoro groups, for instance trifluoroalkyl groups, such as those sold by the company General Electric under the name “FF.150 FLUOROSILICONE FLUID”;

c) hydroxyacylamino groups, such as those described in European Patent Application EP-A-0 342 834, and for example the silicone sold by the company Dow Corning under the name “Q2-8413”;

d) thiol groups, such as the silicones “X 2-8360” from Dow Corning or “GP 72A” and “GP 71” from Genesee;

e) substituted or unsubstituted amino groups, for instance the products sold under the name GP 4 SILICONE FLUID and GP 7100 by the company Genesee or the products sold under the names Q2 8220 and DOW CORNING® 929 or 939 by the company Dow Corning. The substituted amino groups include, for example, C₁-C₄ aminoalkyl or amino(C₁-C₄)alkylamino(C₁-C₄)alkyl groups. The silicones known as amodimethicone and trimethylsilylamodimethicone according to the CTFA name (1997) may, for example, be used;

f) carboxylate groups, for example the products described in European Patent EP 186 507 from Chisso Corporation;

g) hydroxylated groups, for example the polyorganosiloxanes comprising a hydroxyalkyl function, described in Patent Application FR-A-2 589 476;

h) alkoxylated groups comprising at least 12 carbon atoms, such as the product “SILICONE COPOLYMER F 755” from SWS Silicones;

i) acyloxyalkyl groups comprising at least 12 carbon atoms, for example the polyorganosiloxanes described in Patent Application FR-A-2 641 185;

j) quaternary ammonium groups, such as the product “ABIL K 3270” from the company Goldschmidt;

k) amphoteric or betaine groups, such as in the product sold by the company Goldschmidt under the name “ABIL B 9950”;

l) bisulphite groups, such as in the products sold by the company Goldschmidt under the names “ABIL S 201” and “ABIL S 255”;

vii) block copolymers comprising a polysiloxane-polyalkylene linear block as repeat unit; the preparation of such block copolymers used in the context of the present disclosure is described in European Application EP 0 492 657 A1, which is incorporated herein by reference;

viii) grafted silicone polymers comprising a nonsilicone organic backbone constituted of a main organic chain formed from organic monomers which do not comprise silicone, onto which is grafted, within said chain and also, optionally, at least one of its ends, at least one polysiloxane macromonomer; for example those chosen from those described in U.S. Pat. No. 4,963,935, U.S. Pat. No. 4,728,571 and U.S. Pat. No. 4,972,037 and Patent Applications EP-A-0 412 704, EP-A-0 412 707, EP-A-0 640 105 and WO 95/00578, all of which are incorporated herein by reference;

(ix) grafted silicone polymers comprising a polysiloxane backbone grafted with nonsilicone organic monomers, comprising a polysiloxane main chain onto which is grafted, within said chain and also, optionally, at least one of its ends, at least one organic macromonomer which does not comprise silicone; non-limiting examples of such polymers, and also a method for preparing them, are for example described in Patent Applications EP-A-0 582 152, WO 93/23009 and WO 95/03776, all of which are incorporated herein by reference;

(x) or mixtures thereof.

Non-limiting examples of the at least one silicone for use according to the disclosure include non-volatile polyorganopolysiloxanes, and for example polydimethylsiloxane oils or gums which optionally comprise amino, aryl or alkylaryl groups.

The at least one silicone may be present in the compositions of the disclosure in an amount ranging from 0.01% to 20% by weight, and for example from 0.1% to 10% by weight, relative to the total weight of the composition.

According to the present disclosure, the compounds of ceramide type may be, for example, natural or synthetic ceramides and/or glycoceramides and/or pseudoceramides and/or neoceramides.

Compounds of ceramide type are, for example, described in Patent Applications DE442 4530, DE4424533, DE4402929, DE4420736, WO 95/23807, WO 94/07844, EP-A-0646572, WO 95/16665, FR-2-673 179, EP-A-0227994, WO 94/07844, WO 94/24087 and WO 94/10131, all of which are incorporated herein by reference.

Non-limiting examples of the compounds of ceramide type according to the disclosure include:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, and for example N-stearoylphytosphingosine,
2-N-palmitoylaminohexadecane-1,3-diol,
(bis(N-hydroxyethyl-N-cetyl)malonamide),
cetyl acid N-(2-hydroxyethyl)-N-(3-cetyloxy-2-hydroxypropyl)amide,
N-docosanoyl-N-methyl-D-glucamine,
or mixtures of these compounds.

The water-insoluble carboxylic acid esters according to the disclosure are insoluble in water at a concentration of greater than or equal to 0.1% by weight in water at 25° C., i.e., under these conditions, they do not form an isotropic solution transparent to the naked eye.

The esters that can be used in the present disclosure may be, for example, monomeric. They may be, for example, nonionic, nonsilicone esters.

The water-insoluble carboxylic acid esters according to the disclosure may comprise hydroxyl groups.

The total number of carbons of the esters of the disclosure may, for example, range from 12 to 50, such as from 16 to 40, and for example from 16 to 30.

According to the disclosure, the esters used may be, for example, liquid at ambient temperature (25° C.) and at atmospheric pressure (1 atm).

The carboxylic esters are, in at least one embodiment, chosen from:

1) esters of an aliphatic or alicyclic C₃-C₃₀ carboxylic acid and of a C₁-C₃₀ alcohol, at least one of the acid or of the alcohol being branched or comprising at least one carbon-carbon double bond, and

2) esters of a C₇-C₃₀ aromatic acid, the carboxylic function of which is directly linked to the aromatic ring, and of a C₁-C₃₀ alcohol.

The esters of a C₃-C₃₀ carboxylic acid and of a C₁-C₃₀ alcohol, at least one of the acid or of the alcohol being branched or unsaturated, may, for example, be chosen from esters of a C₆-C₂₄ carboxylic acid and of a C₃-C₂₀ alcohol.

The esters according to the disclosure may, for example, be chosen from:

esters of a linear carboxylic acid comprising from 12 to 26 carbon atoms and of a branched alcohol comprising from 3 to 12 carbon atoms,

esters of a linear carboxylic acid comprising from 2 to 12 carbon atoms and of a branched alcohol comprising from 8 to 26 carbon atoms, and

esters of a branched carboxylic acid comprising from 8 to 26 carbon atoms, such as from 8 to 12, and of a branched alcohol comprising from 8 to 26 carbon atoms, such as from 8 to 12.

Non-limiting mention may be made of octyldodecyl behenate; isocetyl behenate; isocetyl lactate; isostearyl lactate; isostearyl octanoate; isocetyl octanoate; isocetyl isostearate; isocetyl laurate; isocetyl stearate; isodecyl octanoate; isodecyl oleate; isononyl isononanoate; isostearyl palmitate; myristyl isostearate; octyl isononanoate; 2-ethylhexyl isononate; octyl isostearate; octyldodecyl erucate; isopropyl palmitate, 2-ethylhexyl palmitate, 2-octyldecyl palmitate, branched alkyl myristates such as isopropyl myristate, t-butyl myristate or 2-octyldodecyl myristate, hexyl isostearate, butyl isostearate, isobutyl stearate; and 2-hexyldecyl laurate.

In at least one embodiment, the acid and the alcohol of the ester are saturated.

Liquid esters of a branched carboxylic acid comprising from 4 to 6 carbon atoms and of an alcohol comprising from 8 to 26 carbon atoms may also be used.

These branched liquid esters according to the disclosure may, for example, be chosen from the following formula:

R₁COOR₂ (VI)

wherein:
R₁ is an optionally monohydroxylated or polyhydroxylated, branched hydrocarbon-based radical comprising from 3 to 5 carbon atoms,
R₂ is an optionally monohydroxylated or polyhydroxylated, linear or branched, hydrocarbon-based radical comprising from 12 to 26 carbon atoms, such as from 16 to 22 carbon atoms.

In at least one embodiment, R₁ is a branched alkyl radical comprising from 3 to 5 carbon atoms, for example a tert-butyl radical. In yet another embodiment, R₂ is a saturated or unsaturated alkyl radical comprising from 12 to 26 carbon atoms, for example branched, and such as chosen from tridecyl, isocetyl, isostearyl, octyldodecyl, and isoarachidyl radicals.

Non-limiting examples of the branched liquid esters include isostearyl neopentanoate (formula VI wherein R₁ is tert-butyl and R₂ is isostearyl), tridecyl neopentanoate, isocetyl neopentanoate, and isoarachidyl neopentanoate.

In at least one embodiment the acid and the alcohol of the ester are saturated. In another embodiment, the alcohol is a monoalcohol (a single hydroxyl function).

Among the esters mentioned above, non-limiting mention may further be made of isopropyl palmitate, 2-ethylhexyl palmitate, 2-octyldecyl palmitate, branched alkyl myristates such as isopropyl myristate, t-butyl myristate or 2-octyldodecyl myristate, hexyl isostearate, butyl isostearate, isobutyl stearate, 2-hexyldecyl laurate, isostearyl neopentanoate, tridecyl neopentanoate and isononyl isononanoate.

The esters of a C₇-C₃₀ aromatic acid and of a C₁-C₃₀ alcohol may be, for example, esters of a C₇-C₁₇ aromatic acid and of a C₁-C₂₀ alcohol. These esters include, for example, C₁₂-C₁₅ alkyl benzoates, isostearyl benzoate, octyldodecyl benzoate, behenyl benzoate or 2-ethylhexyl benzoate.

According to the disclosure, the conditioning agents may present in an amount ranging from 0.001% to 10% by weight, such as from 0.005% to 5% by weight, and for example from 0.01% to 3% by weight, relative to the total weight of the final composition.

The physiologically acceptable medium may be, for example, constituted of water or a mixture of water and cosmetically or dermatologically acceptable solvents such as monoalcohols, polyols or polyol ethers which can be used alone or as a mixture. The water may, for example, be present in amount ranging from 30% to 98% by weight, and for example from 50% to 98% by weight, relative to the total weight of the composition.

Non-limiting mention may be made of monoalcohols such as ethanol or isopropanol, polyols such as diethylene glycol or glycerol, and polyol ethers such as diethylene glycol ethers.

The composition of the disclosure may further comprise at least one additive chosen from sequestering agents, emollients, foam modifiers, dyes, pearlescent agents, moisturizers, anti-dandruff or anti-seborrhoeic agents, anti-hairloss or hair-regrowth agents, suspending agents, fatty acids, fragrances, preservatives, sunscreens, noncationic proteins and protein hydrolysates, vitamins and provitamins, anionic, nonionic or amphoteric polymers, basifying or acidifying agents, humectants, sugars, menthol, nicotinate derivatives, foam stabilizers, propellants, and any other additive conventionally used in the cosmetics field.

The at least one additive may be present in the composition according to the disclosure in an amount ranging from 0 to 40% by weight, relative to the total weight of the composition. The precise amount of each additive depends on its nature and may be readily determined by those skilled in the art.

Of course, those skilled in the art will take care to select the optional at least one additive to be added to the composition according to the disclosure in such a way that the advantageous properties intrinsically associated with the composition in accordance with the disclosure are not, or not substantially, impaired by the addition envisaged.

The compositions in accordance with the disclosure may be, for example, used for washing or treating keratin materials such as the hair, the skin, the eyelashes, the eyebrows, the nails, the lips or the scalp, and, in at least one embodiment, the hair.

The compositions according to the disclosure may, for example, be used as products for washing, caring for, conditioning, retaining the hairstyle or shaping keratin materials such as the hair.

The compositions of the disclosure may be, for example, in the form of shampoos, rinse-out or leave-in conditioners, compositions for permanent-waving, hair-straightening, dyeing or bleaching, or in the form of compositions to be applied before or after a dyeing, bleaching, permanent-waving or hair-straightening operation, or alternatively between the two steps of a permanent-waving or hair-straightening operation.

In at least one embodiment, the compositions are washing and foaming compositions for the hair and/or the skin.

In yet another embodiment, the compositions according to the disclosure are foaming detergent compositions such as shampoos, shower gels, bubble baths and makeup-removing products. In this embodiment of the disclosure, the compositions may comprise at least one detergent surfactant.

The at least one detergent surfactant may be chosen, without distinction, alone or as mixtures, from the anionic, amphoteric or nonionic surfactants, for example, as defined above.

In the compositions in accordance with the disclosure in the form of detergent compositions, such as shampoos, at least one anionic surfactant or mixtures of at least one anionic surfactant and of at least one amphoteric surfactant or of at least one nonionic surfactant may, for example, be used.

In at least one embodiment, the mixture is a mixture comprising at least one anionic surfactant and at least one amphoteric surfactant.

Use may, for example, be made of an anionic surfactant chosen from sodium, triethanolamine or ammonium (C₁₂-C₁₄)alkyl sulphates, sodium, triethanolamine or ammonium (C₁₂-C₁₄)alkyl ether sulphates oxyethylenated comprising 2.2 mol of ethylene oxide, sodium cocoyl isethionate and sodium (C₁₄-C₁₆)-alpha-olefin sulphonate, and mixtures thereof with:

either an amphoteric surfactant such as the amine derivatives known as disodium cocoamphodipropionate or sodium cocoamphopropionate sold, for example, by the company Rhodia Chimie under the trade name “MIRANOL C2M CONC” as an aqueous solution comprising 38% of active material, or under the name MIRANOL C32;

or an amphoteric surfactant of zwitterionic type such as alkylbetaines or alkylamidobetaines, for example the cocobetaine sold under the name “DEHYTON® AB 30” as an aqueous solution comprising 32% of active material by the company Cognis.

Use may be made, for example, of an anionic surfactant chosen from sodium, triethanolamine or ammonium (C₁₂-C₁₄)alkyl sulphates, sodium, triethanolamine or ammonium (C₁₂-C₁₄)alkyl ether sulphates oxyethylenated comprising 2.2 mol of ethylene oxide, sodium cocoyl isethionate and sodium (C₁₄-C₁₆)-alpha-olefin sulphonate, and mixtures thereof with:

either an amphoteric surfactant such as the amine derivatives known as disodium cocoamphodipropionate or sodium cocoamphopropionate sold for example by the company Rhodia Chimie under the trade name “MIRANOL C2M CONC” as an aqueous solution comprising 38% of active material, or under the name MIRANOL C32;

or an amphoteric surfactant of zwitterionic type such as alkylbetaines, for example the cocobetaine sold under the name “DEHYTON® AB 30” as an aqueous solution comprising 32% of active material by the company Cognis and the cocoamidopropylbetaine sold for example by Goldschmidt under the name TEGOBETAINE F50.

The minimum amount of surfactant is that which is just sufficient to confer on the final composition a satisfactory foaming and/or detergent power.

Thus, according to the disclosure, the detergent surfactant may be present in an amount ranging from 3% to 30% by weight, such as from 6% to 25% by weight, and for example from 8% to 20% by weight, relative to the total weight of the final composition.

The foaming power of the compositions according to the disclosure, when characterized by a foam height, may be greater than 75 mm, for example greater than 100 mm, measured according to the modified Ross-Miles method (NF T 73-404/ISO696).

The modifications to the method are as follows:

The measurement is performed at a temperature of 22° C. with osmosed water. The concentration of the solution is 2 g/l. The drop height is 1 m. The amount of composition which is dropped is 200 ml. These 200 ml of composition fall into a measuring cylinder having a diameter of 50 mm and containing 50 ml of the test composition. The measurement is carried out 5 minutes after the flow of the composition has been stopped.

In another embodiment, when the composition is in the form of an optionally rinse-out conditioner, it may comprise at least one cationic surfactant, wherein the concentration of said at least one cationic surfactant may range from 0.1% to 10% by weight, and for example from 0.5% to 5% by weight, relative to the total weight of the composition.

The compositions of the disclosure may also be in the form of compositions for permanent-waving, hair-straightening, dyeing or bleaching, or in the form of rinse-out compositions to be applied before or after a dyeing, bleaching, permanent-waving or hair-straightening operation or alternatively between the two steps of a permanent-waving or hair-straightening operation.

The compositions according to the disclosure may be in the form of aqueous or aqueous-alcoholic lotions for skin care and/or hair care.

The cosmetic compositions according to the disclosure may be in the form of a gel, a milk, a cream, an emulsion, a thickened lotion or a mousse and may be used for the skin, the nails, the eyelashes, the lips and, in at least one embodiment, the hair.

The compositions may be packaged in various forms, for example in vaporizers, pump-dispenser bottles or aerosol containers in order to be able to apply the composition in a vaporized form or in the form of a mousse, Such packaging forms are indicated, for example, when it is desired to obtain a spray, a lacquer or a mousse for treating the hair.

One embodiment of the disclosure is a process for treating keratin materials such as the skin or the hair, comprising: applying a cosmetic composition as defined above to the keratin materials and then optionally rinsing with water after an optional leave-in time.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The disclosure will now be illustrated more fully with the aid of the following examples, which are not limited to the embodiments described. In the subsequent text, AM signifies Active Material.

The examples which follow are intended merely to illustrate the disclosure.

### EXAMPLE 1

The following shampoo composition was prepared:

| | | |
|---|---|---|
| Sodium lauryl ether sulphate (2.2 EO) as an aqueous | 14.9 | g AM |
| solution comprising 30% AM (TEXAPON ® AOS | | |
| 225 UP from Cognis) | | |
| Sodium cocoamidoethyl (N-hydroxyethyl-N- | 2.4 | g AM |
| carboxymethyl) glycinate as an aqueous solution | | |
| comprising 30% AM (MIRANOL C2M CONC. | | |
| NP from Rhodia) | | |
| Copolymer of esters of acrylic or methacrylic acid, of | 1 | g AM |
| (C₁-C₄) dialkylamino(C₁-C₆)alkyl methacrylate, | | |
| of PEG/PPG-30/5 methacrylate, allyl ether, | | |
| of C10-30 alkyl PEG 20-25 ether of hydroxy | | |
| C2-6 alkyl methacrylate crosslinked with ethylene | | |
| glycol dimethacrylate as an emulsion at 20% in water | | |
| (CARBOPOL ® Aqua CC Polymer from Noveon) | | |
| Weakly crosslinked sodium carboxymethyl starch from | 5.65 | g AM |
| potato (PRIMOJEL ® from Avebe) | | |
| Lactic acid | 0.38 | g |
| Disodium salt of ponceau SX (CI: 14,700) | 0.00015 | g |
| Powdered salicylic acid | 0.2 | g |
| Ethyl p-hydroxybenzoate | 0.15 | g |
| Sodium benzoate | 0.5 | g |
| Methyl p-hydroxybenzoate, sodium salt | 0.4 | g |
| Fragrance | 0.5 | g |
| Pure sodium hydroxide or citric acid. 1 H₂O | qs pH | 5.3 |
| Deionized water | qs 100 | g |

This composition had a thickened texture which was stable over time. It was readily applied to and distributed over the hair with a good initiation of lathering. This composition was easily removed with rinsing and gave the treated hair softness, smoothness and sheen.

### EXAMPLE 2

The following shampoo composition was prepared:

| | | |
|---|---|---|
| Sodium lauryl ether sulphate (2.2 EO) as an aqueous | 14.9 | g AM |
| solution comprising 30% AM (TEXAPON ® AOS | | |
| 225 UP from Cognis) | | |
| Sodium cocoamidoethyl (N-hydroxyethyl-N- | 2.4 | g AM |
| carboxymethyl) glycinate as an aqueous solution | | |
| comprising 30% AM (MIRANOL C2M CONC. NP | | |
| from Rhodia) | | |
| Copolymer of esters of acrylic or methacrylic acid, of | 1 | g AM |
| (C₁-C₄)dialkylamino(C₁-C₆)alkyl methacrylate, of PEG/ | | |
| PPG- 30/5 allyl ether, of C10-30 alkyl PEG 20-25 ether | | |
| methacrylate, of hydroxy C2-6 alkyl methacrylate | | |
| crosslinked with ethylene glycol dimethacrylate as an | | |
| emulsion at 20% in water (CARBOPOL ® Aqua | | |
| CC Polymer from Noveon) | | |
| Weakly crosslinked sodium carboxymethyl starch from | 5.65 | g AM |
| potato (PRIMOJEL ® from Avebe) | | |
| Quaternized hydroxyethylcellulose (JR 400 | 0.5 | g |
| from Amerchol) | | |
| Lactic acid | 0.38 | g |
| Disodium salt of ponceau SX (CI: 14,700) | 0.00015 | g |
| Powdered salicylic acid | 0.2 | g |
| Ethyl p-hydroxybenzoate | 0.15 | g |
| Sodium benzoate | 0.5 | g |
| Methyl p-hydroxybenzoate, sodium salt | 0.4 | g |
| Fragrance | 0.5 | g |
| Pure sodium hydroxide or citric acid. 1 H₂O | qs pH | 5.3 |
| Deionized water | qs 100 | g |

This composition had a thickened texture which was stable over time. It was readily applied to and distributed over the hair with a good initiation of lathering. This composition was easily removed with rinsing and gave the treated hair softness, smoothness and sheen.

### EXAMPLE 3

The following shampoo composition was prepared:

| | | |
|---|---|---|
| Sodium lauryl ether sulphate (2.2 EO) as an aqueous | 12.83 | g AM |
| solution comprising 30% AM (TEXAPON ® AOS | | |
| 225 UP from Cognis) | | |
| Sodium cocoamidoethyl (N-hydroxyethyl-N- | 0.6 | g AM |
| carboxymethyl) glycinate as an aqueous solution | | |
| comprising 30% AM (MIRANOL C2M CONC. NP | | |
| from Rhodia) | | |
| Cocoyl betaine as an aqueous solution comprising 30% | 1.57 | g AM |
| AM (DEHYTON ® AB 30 from Cognis) | | |
| Copolymer of esters of acrylic or methacrylic acid, of | 0.6 | g AM |
| (C₁-C₄)dialkylamino(C₁-C₆)alkyl methacrylate, of PEG/ | | |
| PPG-30/5 allyl ether, of C10-30 alkyl PEG 20-25 ether | | |
| methacrylate, of hydroxy C2-6 alkyl methacrylate | | |
| crosslinked with ethylene glycol dimethacrylate as an | | |
| emulsion at 20% in water (CARBOPOL ® Aqua CC | | |
| Polymer from Noveon) | | |
| Maize distarch phosphate (STRUCTURE ® Zea from | 3 | g AM |
| National Starch) | | |
| Ethylene glycol distearate | 2 | g |
| Propylene glycol | 2 | g |
| Lactic acid | 0.38 | g |
| Disodium salt of ponceau SX (CI: 14,700) | 0.00015 | g |
| Powdered salicylic acid | 0.2 | g |
| Ethyl p-hydroxybenzoate | 0.15 | g |
| Sodium benzoate | 0.5 | g |
| Methyl p-hydroxybenzoate, sodium salt | 0.4 | g |
| Fragrance | 0.5 | g |
| Pure sodium hydroxide or citric acid. 1 H₂O | qs pH | 5.3 |
| Deionized water | qs 100 | g |

This composition had a thickened texture which was stable over time. It was readily applied to and distributed over the hair with a good initiation of lathering. This composition was easily removed with rinsing and gave the treated hair softness, smoothness and sheen.

### EXAMPLE 4

The following shampoo composition was prepared:

| | | |
|---|---|---|
| Sodium lauryl ether sulphate (2.2 EO) as an aqueous | 12.83 | g AM |
| solution comprising 30% AM (TEXAPON ® AOS | | |
| 225 UP from Cognis) | | |
| Sodium cocoamidoethyl (N-hydroxyethyl-N- | 0.6 | g AM |
| carboxymethyl) glycinate as an aqueous solution | | |
| comprising 30% AM (MIRANOL C2M CONC. NP | | |
| from Rhodia) | | |
| Cocoyl betaine as an aqueous solution comprising 30% | 1.57 | g AM |
| AM (DEHYTON ® AB 30 from Cognis) | | |
| Copolymer of esters of acrylic or methacrylic acid, of | | |
| (C₁-C₄)dialkylamino(C₁-C₆)alkyl methacrylate, of PEG/ | | |
| PPG-30/5 allyl ether, of C10-30 alkyl PEG 20-25 ether | | |
| methacrylate, of hydroxy C2-6 alkyl methacrylate | | |
| crosslinked with ethylene glycol dimethacrylate as an | 0.6 | g AM |
| emulsion at 20% in water (CARBOPOL ® Aqua CC | | |
| Polymer from Noveon) | | |
| Modified, crosslinked and pregelatinized tapioca starch | 3 | g AM |
| (ULTRA-SPERSE 3 from National Starch) | | |
| Virgin olive oil | 0.5 | g |
| Ethylene glycol distearate | 2 | g |
| Lactic acid | 0.38 | g |
| Disodium salt of ponceau SX (CI: 14,700) | 0.00015 | g |
| Powdered salicylic acid | 0.2 | g |
| Ethyl p-hydroxybenzoate | 0.15 | g |
| Sodium benzoate | 0.5 | g |
| Methyl p-hydroxybenzoate, sodium salt | 0.4 | g |
| Fragrance | 0.5 | g |
| Pure sodium hydroxide or citric acid. 1 H₂O | qs pH | 5.3 |
| Deionized water | qs 100 | g |

This composition had a thickened texture which was stable over time. It was readily applied to and distributed over the hair with a good initiation of lathering. This composition was easily removed with rinsing and gave the treated hair softness, smoothness and sheen.

### EXAMPLE 5

The following shampoo composition was prepared:

| | | |
|---|---|---|
| Sodium lauryl ether sulphate (2.2 EO) as an aqueous | 12.83 | g AM |
| solution comprising 30% AM (TEXAPON ® AOS 225 | | |
| UP from Cognis) | | |
| Sodium cocoamidoethyl (N-hydroxyethyl-N- | 0.6 | g AM |
| carboxymethyl) glycinate as an aqueous solution | | |
| comprising 30% AM (MIRANOL C2M CONC. NP | | |
| from Rhodia) | | |
| Cocoyl betaine as an aqueous solution comprising 30% | 1.57 | g AM |
| AM (DEHYTON ® AB 30 from Cognis) | | |
| Copolymer of esters of acrylic or methacrylic acid, of | 0.6 | g AM |
| (C₁-C₄)dialkylamino(C₁-C₆)alkyl methacrylate, of PEG/ | | |
| PPG-30/5 allyl ether, of C10-30 alkyl PEG 20-25 ether | | |
| methacrylate, of hydroxy C2-6 alkyl methacrylate | | |
| crosslinked with ethylene glycol dimethacrylate as an | | |
| emulsion at 20% in water (CARBOPOL ® Aqua CC | | |
| Polymer from Noveon) | | |
| Modified, crosslinked and pregelatinized tapioca starch | 3 | g AM |
| (ULTRA-SPERSE 3 from National Starch) | | |
| Hydroxypropyl guar trimethylammonium chloride | 0.2 | g |
| (JAGUAR EXCEL from Rhodia) | | |
| Ethylene glycol distearate | 2 | g |
| Lactic acid | 0.38 | g |
| Disodium salt of ponceau SX (CI: 14,700) | 0.00015 | g |
| Powdered salicylic acid | 0.2 | g |
| Ethyl p-hydroxybenzoate | 0.15 | g |
| Sodium benzoate | 0.5 | g |
| Methyl p-hydroxybenzoate, sodium salt | 0.4 | g |
| Fragrance | 0.5 | g |
| Pure sodium hydroxide or citric acid. 1 H₂O | qs pH | 5.3 |
| Deionized water | qs 100 | g |

This composition had a thickened texture which was stable over time. It was readily applied to and distributed over the hair with a good initiation of lathering. This composition was easily removed with rinsing and gave the treated hair softness, smoothness and sheen.

### EXAMPLE 6

The following rinse-out conditioner composition was prepared:

| Stearyl alcohol | 2 | g |
|---|---|---|
| Copolymer of esters of acrylic or methacrylic acid, of | 0.8 | g AM |
| (C₁-C₄)dialkylamino(C₁-C₆)alkyl methacrylate, of PEG/ | | |
| PPG-30/5 allyl ether, of C10-30 alkyl PEG 20-25 ether | | |
| methacrylate, of hydroxy C2-6 alkyl methacrylate | | |
| crosslinked with ethylene glycol dimethacrylate as an | | |
| emulsion at 20% in water (CARBOPOL ® Aqua CC | | |
| Polymer from Noveon) | | |
| Behenyltrimethylammonium chloride as an aqueous | 1.6 | g AM |
| solution comprising 80% of AM (GENAMIN KDMP from | | |
| Clariant) | | |
| Quaternium-87 as a solution comprising 75% AM in | 2.51 | g AM |
| propylene glycol (VARISOFT W 575 PG from | | |
| Goldschmidt) Polydimethylsiloxane comprising aminoethyl- | 0.8 | g AM |
| iminopropyl groups as a nonionic aqueous emulsion | | |
| comprising 20% of AM (SME 253 from Momentive | | |
| Performance Materials) | | |
| Hydroxypropylated and pregelatinized maize distarch | 2 | g AM |
| phosphate (STRUCTURE ® Zea from National Starch) | | |
| Candelilla wax (Candelilla wax SP75 from Strahl & Pitsch) | 0.4 | g |
| Fragrance, preservatives | qs | |
| Lactic acid qs | pH 4 | |
| Water qs | 100 | g |

This stable composition had a pleasant texture and was applied to highly sensitized hair. It was easily removed during rinsing. The cosmetic properties of the treated hair (disentangling, smoothness, manageability) were excellent and homogeneous from the roots to the ends of the hair. The ends were not split.

Between two applications, the hair remained soft, manageable and smooth.

### EXAMPLE 7

The following rinse-out conditioner composition was prepared:

| Copolymer of esters of acrylic or methacrylic acid, of | 0.25 | g AM |
|---|---|---|
| (C₁-C₄)dialkylamino(C₁-C₆)alkyl methacrylate, of PEG/ | | |
| PPG-30/5 allyl ether, of C10-30 alkyl PEG 20-25 ether | | |
| methacrylate, of hydroxy C2-6 alkyl methacrylate | | |
| crosslinked with ethylene glycol dimethacrylate as an | | |
| emulsion at 20% in water (CARBOPOL ® Aqua CC | | |
| Polymer from Noveon) | | |
| Behenyltrimethylammonium chloride as an aqueous | 1.6 | g AM |
| solution comprising 80% of AM (GENAMIN KDMP from | | |
| Clariant) | | |
| Cationic emulsion comprising 67% AM of | 0.8 | g |
| polydimethylsiloxane comprising alpha,omega-vinyl | | |
| groups/polydimethylsiloxane comprising alpha,omega- | | |
| hydrogeno groups copolymer (DC-1997 from | | |
| Dow Corning) | | |
| 2-hydroxypropylether guar (JAGUAR HP 105 | 0.3 | g |
| from Rhodia) | | |
| Hydroxypropylated and pregelatinized maize distarch | 7.52 | g AM |
| phosphate (STRUCTURE ® Zea from National Starch) | | |
| Mica-titanium oxide (67.5/32.5) | 0.4 | g |
| Fragrance, preservatives | qs | |
| Lactic acid qs | pH 4 | |
| Water qs | 100 | g |

This stable composition had a pleasant texture and was applied to highly sensitized hair. It was easily removed during rinsing. The cosmetic properties of the treated hair (disentangling, smoothness, manageability) were excellent and homogeneous from the roots to the ends of the hair. The ends were not split.

Between two applications, the hair remained soft, manageable and smooth.

What is claimed is:

1. A cosmetic composition comprising, in a cosmetically acceptable medium:
(i) at least one cationic polymer which is produced by polymerization of a mixture of monomers comprising:
a) at least one vinyl monomer substituted with at least one amino group,
b) at least one hydrophobic nonionic vinyl monomer chosen from those of formulae (I) and (II):
CH₂═C(X)Z, (I)
CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, —N-(2-pyrrolidonyl), —N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group; and
each R¹ independently is chosen from C₁-C₃₀ alkyl group, a hydroxylated C₂-C₃₀ alkyl group, and a halogenated C₁-C₃₀ alkyl group;
c) at least one associative vinyl monomer, and
d) at least one hydroxylated nonionic vinyl monomer; and
(ii) at least one starch.

2. The composition according to claim 1, wherein the at least one vinyl monomer substituted with at least one amino group is chosen from:
mono(C₁-C₄)alkylamino(C₁-C₈)alkyl (meth)acrylates,
di(C₁-C₄)alkylamino(C₁-C₈)alkyl (meth)acrylates,
mono(C₁-C₄)alkylamino(C₁-C₈)alkyl(meth)acrylamides,
di(C₁-C₄)alkylamino(C₁-C₈)alkyl(meth)acrylamides,
heterocyclic (meth)acrylamides comprising a nitrogen atom,
heterocyclic (meth)acrylates comprising a nitrogen atom, and mixtures thereof.

3. The composition according to claim 2, wherein the di(C₁-C₄)alkylamino(C₁-C₈)alkyl (meth)acrylate is chosen from di(C₁-C₄)alkylamino(C₁-C₆)alkyl (meth)acrylates.

4. The composition according to claim 1, wherein the at least one vinyl monomer substituted with at least one amino group is chosen from:
mono- or di(C₁-C₄ alkyl)amino(C₁-C₄ alkyl) (meth)acrylates;
mono- or di(C₁-C₄ alkyl)amino(C₁-C₄ alkyl)(meth)acrylamides;
(meth)acrylamides or (meth)acrylates comprising a heterocyclic group comprising a nitrogen atom; and
nitrogenous heterocycles comprising at least one vinyl group.

5. The composition according to claim 4, wherein the mono- or di(C₁-C₄ alkyl)amino(C₁-C₄ alkyl)(meth)acrylates are chosen from 2-(N,N-dimethylamino)ethyl (meth)acrylate, 3-(N,N-dimethylamino)propyl (meth)acrylate, 4-(N,N-dimethylamino)butyl (meth)acrylate, (N,N-dimethylamino)-t-butyl (meth)acrylate, 2-(N,N-diethylamino)ethyl (meth)acrylate, 3-(N,N-diethylamino)propyl (meth)acrylate, 4-(N,N-diethylamino)butyl (meth)acrylate, 2-(N,N-dipropylamino)ethyl (meth)acrylate, 3-(N,N-dipropylamino)propyl (meth)acrylate, and 4-(N,N-dipropylamino)butyl (meth)acrylate.

6. The composition according to claim 4, wherein the mono- or di(C₁-C₄ alkyl)amino(C₁-C₄ alkyl)(meth)acrylamides are chosen from N′-(2-N,N-dimethylamino)ethyl(meth)acrylamide and N′-(3-N,N-dimethylamino)propylacrylamide.

7. The composition according to claim 4, wherein the (meth)acrylamides or (meth)acrylates comprising a heterocyclic group comprising a nitrogen atom are chosen from N-(2-pyridyl)acrylamide, N-(2-imidazolyl)methacrylamide, 2-(4-morpholinyl)ethyl methacrylate, 2-(4-morpholinyl)ethyl acrylate, N-(4-morpholinyl)methacrylamide, and N-(4-morpholinyl)acrylamide.

8. The composition according to claim 4, wherein the nitrogenous heterocycles comprising at least one vinyl group are chosen from 2-vinylpyridine and 4-vinylpyridine.

9. The composition according to claim 1, wherein the at least one vinyl monomer substituted with at least one amino group is present in amount ranging from 10% to 70% by weight, relative to the total weight of the mixture of monomers.

10. The composition according to claim 9, wherein that the at least one vinyl monomer substituted with at least one amino group is present in amount ranging from 20% to 60% by weight, relative to the total weight of the mixture of monomers.

11. The composition according to claim 1, wherein the at least one hydrophobic nonionic vinyl monomer is chosen from C₁-C₃₀ alkyl (meth)acrylates, (C₁-C₃₀ alkyl)(meth)acrylamides, styrene, substituted styrenes, vinyl esters, unsaturated nitrites, and unsaturated silanes.

12. The composition according to claim 1, wherein the at least one hydrophobic nonionic vinyl monomer is present in an amount ranging from 20% to 80% by weight, relative to the total weight of the mixture of monomers.

13. The composition according to claim 12, wherein the at least one hydrophobic nonionic vinyl monomer is present in an amount ranging from 20% to 70% by weight, relative to the total weight of the mixture of monomers.

14. The composition according to claim 1, wherein the at least one associative vinyl monomer is chosen from the compounds of formula (III): 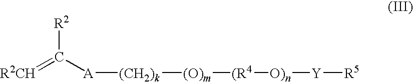
wherein:
each R² is independently chosen from a hydrogen atom, a methyl group, a —C(O)OH group, and a —C(O)OR³ group;
R³ is a C₁-C₃₀ alkyl;
A is chosen from —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, and —CH₂CH₂—NHC(O)— groups;
Ar is a divalent aryl group;
E is chosen from a hydrogen atom, and a methyl group;
z is an integer ranging from 0 to 1;
k is an integer ranging from 0 to 30;
m is an integer ranging from 0 to 1, with the provisos that, when k is 0, then m is 0, and when k is an integer ranging from 1 to 30, then m is 1;
(R⁴—O)ₙ is a polyoxyalkylene, which is a homopolymer, a random copolymer, or a block copolymer, comprising C₂-C₄ oxyalkylene units;
R⁴ is chosen from C₂H₄, C₃H₈, C₄H₈, and mixtures thereof;
n is an integer ranging from 5 to 250;
Y is chosen from —R⁴O—, —R⁴NH—, —C(O)—, —C(O)NH—, —R⁴NHC(O)NH—, and —C(O)NH C(O)—;
R⁵ is a substituted or unsubstituted alkyl chosen from linear C₈-C₄₀ alkyls, branched C₈-C₄₀ alkyls, C₈-C₄₀ alicyclics, phenyls substituted with a C₂-C₄₀ alkyl group, C₂-C₄₀ alkyls substituted with an aryl group, and C₈-C₈₀ complex esters, wherein the R⁵ alkyl group optionally comprises at least one substituent chosen from hydroxyl, alkoxy, and halo groups.

15. The composition according to claim 14, wherein the at least one associative vinyl monomer is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tri(1′-phenylethyl)phenyl (meth)acrylates, polyethoxylated (meth)acrylates of hydrogenated castor oil, polyethoxylated canola (meth)acrylates, polyethoxylated (meth)acrylates of cholesterol, and mixtures thereof, wherein the polyethoxylated portion of the monomer comprises from 5 to 100 ethylene oxide units.

16. The composition according to claim 15, wherein the polyethoxylated portion of the monomer comprises from 15 to 60 ethylene oxide units.

17. The composition according to claim 1, wherein the at least one associative vinyl monomer is present in an amount ranging from 0.001% to 25% by weight, relative to the total weight of the mixture of monomers.

18. The composition according to claim 17, wherein the at least one associative vinyl monomer is present in an amount ranging from 0.01% to 15% by weight, relative to the total weight of the mixture of monomers.

19. The composition according to claim 1, wherein the mixture of monomers further comprises at least one semi-hydrophobic surfactant vinyl monomer chosen from compounds of formula (IV) or (V): 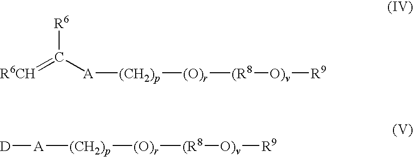
wherein:
each R⁶ independently is chosen from a hydrogen atom, a C₁-C₃₀ alkyl, —C(O)OH, and —C(O)OR⁷;
R⁷ is a C₁-C₃₀ alkyl;
A is chosen from —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, and —CH₂CH₂NHC(O)— groups;
Ar is a divalent aryl group;
E is chosen from a hydrogen atom and a methyl group;
z is an integer ranging from 0 to 1;
p is an integer ranging from 0 to 30;
r is an integer ranging from 0 to 1, with the provisos that, when p is 0, then r is 0, and when p is an integer ranging from 1 to 30, r is 1;
(R₈—O)ᵥ is a polyoxyalkylene which is a homopolymer, a random copolymer or a block copolymer comprising C₂-C₄ oxyalkylene units, wherein R⁸ is chosen from C₂H₄, C₃H₆, C₄H₈, and mixtures thereof, and v is an integer ranging from 5 to 250;
R⁹ is chosen from a hydrogen atom and a C₁-C₄ alkyl; and
D is chosen from a C₈-C₃₀ alkenyl and a C₈-C₃₀ alkenyl substituted with a carboxyl group.

20. The composition according to claim 19, wherein the at least one semi-hydrophobic surfactant vinyl monomer is chosen from:
CH₂═CH—O(CH₂)ₐO(C₃H₆O)_{b}(C₂H₄O)_{c}H, and
CH₂═CHCH₂O(C₃H₆O)_{d}(C₂H₄O)ₑH;
wherein
a is an integer ranging from 2 to 4;
b is an integer ranging from 1 to 10;
c is an integer ranging from 5 to 50;
d is an integer ranging from 1 to 10; and
e is an integer ranging from 5 to 50.

21. The composition according to claim 19, wherein the at least one semi-hydrophobic surfactant vinyl monomer is present in an amount ranging from 0% to 25% by weight, relative to the total weight of the mixture of monomers.

22. The composition according to claim 21, wherein the at least one semi-hydrophobic surfactant vinyl monomer is present in an amount ranging from 0.1% to 10% by weight, relative to the total weight of the mixture of monomers.

23. The composition according to claim 1, wherein the at least one hydroxylated nonionic vinyl monomer is chosen from C₁-C₆ hydroxyalkyl (meth)acrylates, C₁-C₄ hydroxyalkyl (meth)acrylamides, and mixtures thereof.

24. The composition according to claim 23, wherein the at least one hydroxylated nonionic vinyl monomer is 2-hydroxyethyl methacrylate.

25. The composition according to claim 1, wherein the at least one hydroxylated nonionic vinyl monomer is present in an amount ranging from 0.01% to 10% by weight, relative to the total weight of the mixture of monomers.

26. The composition according to claim 1, wherein the mixture of monomers further comprises at least one crosslinking monomer.

27. The composition according to claim 1, wherein the mixture of monomers further comprises at least one chain-transfer agent.

28. The composition according to claim 1, wherein the mixture of monomers further comprises at least one polymeric stabilizer.

29. The composition according to the claim 1, wherein the mixture of monomers comprises, relative to the total weight of the mixture of monomers:
a) from 20% to 60% by weight of the at least one vinyl monomer substituted with at least one amino group,
b) from 20% to 70% by weight of the at least one hydrophobic nonionic vinyl monomer,
c) from 0.01% to 15% by weight of the at least one associative vinyl monomer, and
d) from 0.01% to 10% by weight of the at least one hydroxylated nonionic vinyl monomer.

30. The composition according to claim 19, wherein the mixture of monomers comprises, relative to the total weight of the mixture of monomers from 0.1% to 10% by weight of the at least one semi-hydrophobic surfactant vinyl monomer.

31. The composition according to claim 26, wherein the mixture of monomers comprises, relative to the total weight of the mixture of monomers from 0.001% to 5% by weight of at least one crosslinking monomer.

32. The composition according to claim 27, wherein the mixture of monomers comprises, relative to the total weight of the mixture of monomers from 0.001% to 10% by weight of at least one chain-transfer agent.

33. The composition according to claim 28, wherein the mixture of monomers comprises, relative to the total weight of the mixture of monomers from 0 to 2% by weight of at least one polymeric stabilizer.

34. The composition according to claim 1, wherein said mixture of monomers comprises:
a di(C₁-C₄ alkyl)amino(C₁-C₆ alkyl)methacrylate,
at least one (meth)acrylic acid C₁-C₃₀ alkyl esters,
a polyethoxylated C₁₀-C₃₀ alkyl methacrylate comprising from 20 to 30 mol of ethylene oxide,
a polyethylene glycol/polypropylene glycol 30/5 alkyl ether,
a hydroxy(C₂-C₆ alkyl)methacrylate, and
an ethylene glycol dimethacrylate.

35. The composition according to claim 1, wherein the at least one cationic polymer (i) is present in an amount ranging from 0.01% to 10% by weight, relative to the total weight of the final composition.

36. The composition according to claim 35, wherein the at least one cationic polymer (i) is present in an amount ranging from 0.1% to 1% by weight, relative to the total weight of the final composition.

37. The composition according to claim 1, wherein the at least one starch is chosen from maize, rice, cassava, barley, potato, wheat, sorghum, and pea starches.

38. The composition according to claim 1, wherein the at least one starch is modified chemically and/or physically.

39. The composition according to claim 1, wherein the at least one starch is modified by at least one of the reactions chosen from: pregelatinization, oxidation, crosslinking, esterification, etherification, amidation, and heat treatments.

40. The composition according to claim 39, wherein the at least one starch is modified by at least one esterification.

41. The composition according to claim 1, wherein the at least one starch is chosen from starch phosphates.

42. The composition according to claim 1, wherein the at least one starch is chosen from amphoteric starches.

43. The composition according to claim 1, wherein the at least one starch is present in an amount ranging from 0.1% to 20% by weight, relative to the total weight of the final composition.

44. The composition according to claim 43, wherein the at least one starch is present in an amount ranging from 0.5% to 15% by weight, relative to the total weight of the final composition.

45. The composition according to claim 1, wherein the composition further comprises at least one surfactant chosen from anionic, amphoteric, nonionic and cationic surfactants, and mixtures thereof.

46. The composition according to claim 1, wherein the composition further comprises at least one conditioning agent.

47. The composition according to claim 46, wherein the conditioning agent is a silicone.

48. The composition according to claim 46, wherein the conditioning agent is a cationic polymer.

49. The composition according to claim 1, wherein the composition is a foaming detergent composition.

50. The composition according to claim 49, wherein the foaming detergent composition is chosen from shampoos, shower gels, makeup-removing products, and bubble baths.

51. The composition according to claim 1, wherein the composition is a rinse-out or leave-in conditioner composition.

52. A method of using a composition as a shampoo, lotion or conditioner, the method comprises: applying the composition as a shampoo, lotion or conditioner, wherein the composition comprises, in a cosmetically acceptable medium:
(i) at least one cationic polymer which is produced by polymerization of a mixture of monomers comprising:
a) at least one vinyl monomer substituted with at least one amino group,
b) at least one hydrophobic nonionic vinyl monomer chosen from those of formulae (I) and (II):
CH₂═C(X)Z, (I)
CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, —N-(2-pyrrolidonyl), —N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group; and
each R¹ independently is chosen from C₁-C₃₀ alkyl group, a hydroxylated C₂-C₃₀ alkyl group, and a halogenated C₁-C₃₀ alkyl group;
c) at least one associative vinyl monomer, and
d) at least one hydroxylated nonionic vinyl monomer; and
(ii) at least one starch.

53. A cosmetic treatment process for keratin materials, wherein the process comprises: applying, to said materials, a composition comprising, in a cosmetically acceptable medium:
(i) at least one cationic polymer which is produced by polymerization of a mixture of monomers comprising:
a) at least one vinyl monomer substituted with at least one amino group,
b) at least one hydrophobic nonionic vinyl monomer chosen from those of formulae (I) and (II):
CH₂═C(X)Z, (I)
CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, —N-(2-pyrrolidonyl), —N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group; and
each R¹ independently is chosen from C₁-C₃₀ alkyl group, a hydroxylated C₂-C₃₀ alkyl group, and a halogenated C₁-C₃₀ alkyl group;
c) at least one associative vinyl monomer, and
d) at least one hydroxylated nonionic vinyl monomer; and
(ii) at least one starch;
and then optionally rinsing with water, after an optional leave-in time.

54. The cosmetic treatment process according to claim 52, wherein the keratin materials are the hair.
